(19) United States
(12) Patent Application Publication
(10) Pub. No.: US 2010/0274161 A1
(43) Pub. Date: Oct. 28, 2010

(54) IMPLOSION TECHNIQUES FOR ULTRASOUND

(75) Inventors: Haim Azhari, Misgav (IL); Liat Tsoref, Tel Aviv (IL); Yossi Gross, Moshav Mazor (IL); Jacob Benarie, Haifa (IL)

Correspondence Address: SUGHRUE MION, PLLC, 2100 PENNSYLVANIA AVENUE, N.W., SUITE 800, WASHINGTON, DC, 20037, US

(73) Assignee: Slender Medical, Ltd., Herzliya (IL)

(21) Appl. No.: 12/682,748

(22) PCT Filed: Oct. 22, 2008
(86) PCT No.: PCT/IL08/01390

§ 371 Date: Jul. 12, 2010

Related U.S. Application Data
(60) Provisional application No. 60/999,139, filed on Oct. 15, 2007.

(51) Int. Cl. A61B 17/225

(52) U.S. Cl. 601/4

(57) ABSTRACT

A housing (50) is placed on skin of a subject, and draws at least a portion of the skin within at least a part of the housing. The housing includes at least first and second support structures (34), placed in contact with a surface of skin surrounding the portion of the skin, the first support structure having a first concave surface (153) and the second support structure having a second concave surface (153) that faces the first concave surface. The apparatus includes one or more first ultrasound transducers (160) coupled to the first support structure and one or more second ultrasound transducers coupled to the second support structure. The apparatus also includes a control unit which drives the first and second ultrasound transducers to induce an implosion wave in a target region of the skin. Other embodiments are also described.

### CROSS-REFERENCES TO RELATED APPLICATIONS

This application:

(a) claims priority from U.S. Provisional Patent Application 60/999,139 to Azhari et al., entitled, “Implosion techniques for ultrasound,” filed Oct. 15, 2007, and

(b) is a continuation-in-part of:

PCT Patent Application Publication WO 07/102,161 to Azhari et al., entitled, “A device for ultrasound monitored tissue treatment,” filed on Mar. 8, 2007, which claims priority from:

U.S. Provisional Patent Application 60/780,772 to Azhari et al., entitled, “A method and system for lypolysis and body contouring,” filed Mar. 9, 2006;

U.S. Provisional Patent Application 60/809,577 to Azhari et al., entitled, “A device for ultrasound monitored tissue treatment,” filed May 30, 2006;

U.S. Provisional Patent Application 60/860,635 to Azhari et al., entitled, “Cosmetic tissue treatment using ultrasound,” filed Nov. 22, 2006;

U.S. Regular patent application Ser. No. 11/651,198 to Azhari et al., entitled, “A device for ultrasound monitored tissue treatment,” filed Jan. 8, 2007; and

U.S. Regular patent application Ser. No. 11/653,115 to Azhari et al., entitled, “A method and system for lipolysis and body contouring,” filed Jan. 12, 2007.

Each of the above applications is incorporated herein by reference.

### FIELD OF THE INVENTION

The present invention relates in general to tissue treatment by application of energy thereto, and specifically to the generation of ultrasound waves that cause implosion.

### BACKGROUND OF THE INVENTION

Systems for applying energy to biological tissue are well known. Such energy application may be intended to heal injured tissue, ablate tissue, or improve the appearance of tissue. Energy may be applied in different forms, such as radiofrequency, laser, or ultrasound.

Implosion waves are known in the art for use in atomic weaponry devices in which a sphere of fissionable material is suddenly compressed into a smaller size and thus a greater density. The core of an implosion-type atomic bomb consists of a sphere or a series of concentric shells of fissionable material surrounded by a jacket of high explosives, which, being simultaneously detonated, implode the fissionable material under enormous pressures into a denser mass that immediately achieves criticality.

PCT Patent Publication WO 07/102,161 to Azhari et al., which is incorporated herein by reference, describes apparatus for lipolysis and body contouring of a subject. The apparatus includes a housing adapted for placement on tissue of the subject. The apparatus also includes a plurality of acoustic elements disposed at respective locations with respect to the housing, including at least a first and a second subset of the acoustic elements, wherein the first subset is configured to transmit energy in a plane defined by the housing, such that at least a portion of the transmitted energy reaches the second subset. Other embodiments are also described.

PCT Publication WO 06/018837 to Azhari et al., which is incorporated herein by reference, describes a method of damaging a target tissue of a subject. The method is described as comprising: (a) imaging a region containing the target tissue; (b) determining a focal region of a damaging radiation; (c) positioning the focal region onto the target tissue; and (d) damaging the target tissue by an effective amount of the damaging radiation. The determination of the focal region is described as being performed by delivering to the region bursts of ultrasonic radiation from a plurality of directions and at a plurality of different frequencies, and passively scanning the region so as to receive from the region ultrasonic radiation having at least one frequency other than the plurality of different frequencies.

PCT Publication WO 01/92846 to Azhari et al., relevant portions of which are incorporated herein by reference, describes a system for the localization of target objects using acoustic signals. The system comprises an acoustic transducer; acoustic reflecting means; processing means and output means. The transducer is adapted to transmit acoustic signals to a target object, receive superposed echoes from the target object; directly from the target object and indirectly, reflected by said acoustic reflecting means, and transmit an electrical signal corresponding to the received superposed acoustic signal to said processing means. The processing means is adapted to compute the position of the target object and output the position through said output means.

U.S. Pat. No. 6,406,429 to Bar-Cohen, relevant portions of which are incorporated herein by reference, describes apparatus and method for early detection of cystic structures indicative of ovarian and breast cancers. The apparatus uses ultrasonic wave energy at a unique resonance frequency for inducing cavitation in cystic fluid characteristic of cystic structures in the ovaries associated with ovarian cancer, and in cystic structures in the breast associated with breast cancer. Induced cavitation bubbles in the cystic fluid implode, creating what are described as “implosion waves” which are detected by ultrasonic receiving transducers attached to the abdomen of the patient. Triangulation of the ultrasonic receiving transducers enables the received signals to be processed and analyzed to identify the location and structure of the cyst.

U.S. Pat. No. 4,608,222 to Brueckner, relevant portions of which are incorporated herein by reference, describes, a method of achieving the controlled release of thermonuclear energy by illuminating a minute, solid density, hollow shell of a mixture of material such as deuterium and tritium with a high intensity, uniformly converging laser wave to effect an extremely rapid build-up of energy in inwardly traveling shock waves to implode the shell creating thermonuclear conditions causing a reaction of deuterons and tritons and a resultant high energy thermonuclear burn. Utilizing the resulting energy as a thermal source and to breed tritium or plutonium. A laser source is also provided wherein the flux level is increased with time to reduce the initial shock heating of fuel and provide maximum compression after implosion; and, in addition, computations and an equation are provided to enable the selection of a design having a high degree of stability and a dependable fusion performance by establishing a proper relationship between the laser energy input and the size and character of the selected material for the fusion capsule.

U.S. Pat. No. 6,645,162 to Friedman et al., relevant portions of which are incorporated herein by reference, describes cells that are destroyed within a subcutaneous tissue region using a transducer disposed externally adjacent to a patient's skin. The transducer emits acoustic energy that is focused at a linear focal zone within the tissue region, the acoustic energy having sufficient intensity to rupture cells within the focal zone while minimizing heating. The transducer may include one or more transducer elements having a partial cylindrical shape, a single planar transducer element coupled to an acoustic lens, or a plurality of linear transducer elements disposed adjacent one another in an arcuate or planar configuration. The transducer may include detectors for sensing cavitation occurring with the focal zone, which is correlated to the extent of cell destruction. A frame may be provided for controlling movement of the transducer along the patient's skin, e.g., in response to the extent of cell destruction caused by the transducer.

United States Patent Application 2006/0058707 to Barthe et al., relevant portions of which are incorporated herein by reference, describes a method and system for ultrasound treatment utilizing a multi-directional transducer to facilitate treatment, such as therapy and/or imaging or other tissue parameter monitoring, in two or more directions. In accordance with an exemplary embodiment, a multi-directional transducer comprises a transduction element configured to provide ultrasound energy, such as radiation, acoustical energy, heat energy, imaging, positional information and/or tissue parameter monitoring signals in two or more directions. The transduction element can comprise various materials for providing ultrasound energy or radiation, such as piezoelectric materials, with and without matching layers. In addition, the transduction element can be configured for substantially uniform, focused and/or defocused radiation patterns, as well as for single, multiple-element and/or multiple-element array configurations. In addition, an exemplary multi-directional transducer can comprise multiple elements, either side by side, stacked or in an array.

United States Patent Application 2005/0049543 to Anderson et al., relevant portions of which are incorporated herein by reference, describes a method, device, and system for modifying or destroying selected tissue, by selecting an area of tissue for treatment, collecting the area between a plurality of energy transmitting elements, applying an electric current and/or electromagnetic radiation between the energy transmitting elements, and applying the electric current and/or electromagnetic radiation until, for example, the cells are modified or destroyed. Cooling may be applied to prevent unwanted modification. Conducting mediums may be applied to control tissue modification. Embodiments may be used for treatment of fat cells, acne, lesions, tattoo removals etc.

U.S. Pat. No. 7,258,674 to Cribbs et al., relevant portions of which are incorporated herein by reference, describes a system for the destruction of adipose tissue utilizing high intensity focused ultrasound (HIFU) within a patient's body. The system comprises a controller for data storage and the operation and control of a plurality of elements. One element is a means for mapping a human body to establish three dimensional coordinate position data for existing adipose tissue. The controller is able to identify the plurality of adipose tissue locations on said human body and establish a protocol for the destruction of the adipose tissue. A HIFU transducer assembly having one or more piezoelectric element(s) is used along with at least one sensor wherein the sensor provides feed back information to the controller for the safe operation of the piezoelectric element(s). The sensor is electronically coupled to the controller, and the controller provides essential treatment command information to one or more piezoelectric element(s) based on positioning information obtained from the three dimensional coordinate position data.

U.S. Pat. No. 6,500,141 to Irion et al., relevant portions of which are incorporated herein by reference, describes an apparatus for treating body tissue, in particular superficial soft tissue, with ultrasound, comprising an ultrasonic generation unit and an applicator, by means of which the ultrasound can be irradiated from an applicator surface facing the body surface from outside through the body surface into the body tissue. A suction apparatus for sucking in the body surface against the applicator surface is provided. An apparatus for treating body tissue including superficial soft tissue, with ultrasound, is described as comprising an ultrasonic generation unit and an applicator having an applicator surface facing the body surface from which the ultrasound can be irradiated through the body surface into the body tissue. A suction apparatus is provided for taking in the body surface against the applicator surface which is curved inwardly.

U.S. Pat. No. 5,601,526 to Chapelon et al., relevant portions of which are incorporated herein by reference, describes a method and apparatus for performing therapy using ultrasound. The apparatus is described as using a treatment device having at least one piezoelectric transducer element to supply ultrasonic waves focused onto a focal point or region that determines the tissue zone submitted to therapy. The treatment device, which is controlled by a control device, supplies two types of ultrasonic waves, the first one being thermal waves that produce a predominantly thermal effect on the tissue being treated and the second one being cavitation waves that produce a predominantly cavitation effect on the tissue to be treated. A therapy method is described, using ultrasound for the purpose of destroying a target. The target includes tissue, which may be located inside a body of a mammal. Ultrasonic waves are focused onto a focal point or region. A tissue zone to be submitted to the therapy is determined. Ultrasonic waves are supplied to the target. The ultrasonic waves of two types: thermal waves, for producing a predominantly thermal effect on tissue to be treated, and cavitation waves, for producing a predominantly cavitation effect on the tissue to be treated. The two types of waves are applied for a time sufficient to effect therapy by destroying at least a portion of the tissue, and the thermal ultrasonic waves are supplied at least at a beginning of treatment. In an embodiment, the ultrasonic waves are supplied after an adjustable predetermined time interval for allowing preheating of the tissue to be treated.

U.S. Pat. No. 5,665,053 to Jacobs, relevant portions of which are incorporated herein by reference, describes an endermology body massager having at least two rollers spaced from each other in a parallel configuration. The rollers rotate in the same direction and are rotatably mounted on movable axes. A vacuum source is connected to the chamber that houses the rollers. The vacuum source facilitates the suction of the skin between the rollers and helps bring the rollers closer to each other during operation. The rollers or housing have ultrasound generators that are selectively controlled by the operator. In a first embodiment, the ultrasound generators are located within the rollers. In the second embodiment, the ultrasound generators are disposed in the housing around the rollers. Therefore, a controlled and combined endermology with ultrasound treatment can be achieved.

U.S. Pat. No. 6,438,424 to Knowlton, relevant portions of which are incorporated herein by reference, describes apparatus to modify a skin surface or a soft tissue structure underlying the skin surface including a template with a mechanical force application surface and a receiving opening to receive a body structure. The mechanical force application surface is configured to receive the body structure and apply pressure to the soft tissue structure. An energy delivery device is coupled to the template. The energy delivery device is configured to deliver sufficient energy to the template to form a template energy delivery surface.

US Patent Application Publication 2004/0039312 to Hillstead et al., relevant portions of which are incorporated herein by reference, describes a system for the destruction of adipose tissue utilizing high intensity focused ultrasound (HIFU) within a patient's body. The system is described as comprising a controller for data storage and the operation and control of a plurality of elements. One element is described as a means for mapping a human body to establish three dimensional coordinate position data for existing adipose tissue. The controller is able to identify the plurality of adipose tissue locations on said human body and establish a protocol for the destruction of the adipose tissue. A HIFU transducer assembly having one or more piezoelectric element(s) is used along with at least one sensor, wherein the sensor provides feedback information to the controller for the safe operation of the piezoelectric element(s). The sensor is electronically coupled to the controller, and the controller provides essential treatment command information to one or more piezoelectric element(s) based on positioning information obtained from the three dimensional coordinate position data.

U.S. Pat. No. 6,113,558 to Rosenschein et al., relevant portions of which are incorporated herein by reference, describes apparatus and method for the application of ultrasound to a location within the body. The apparatus can operate at a pulse duration below about 100 milliseconds and in the range 0.1 milliseconds to 100 milliseconds and a pulse repetition period below about 1 second and in the range of 1 millisecond to 1 second. Duty ratios over 5 and over 8 are also described. Therapeutic applications of ultrasound such as for assisting in the treatment of medical conditions such as cancer and/or other ailments are also described.

U.S. Pat. No. 6,607,498 to Eshel, relevant portions of which are incorporated herein by reference, describes a method and apparatus for producing lysis of adipose tissue underlying the skin of a subject, by (a) applying an ultrasonic transducer to the subject's skin to transmit therethrough ultrasonic waves focused on the adipose tissue, and (b) electrically actuating the ultrasonic transducer to transmit ultrasonic waves to produce cavitational lysis of the adipose tissue without damaging non-adipose tissue.

U.S. Pat. Nos. 5,743,863 and 5,573,497 to Chapelon, relevant portions of which are incorporated herein by reference, describe a high-energy ultrasound therapy method and apparatus. The apparatus comprises a therapy device with at least one ultrasound therapy transducer element and a signal generator supplying an electronic signal to said ultrasound transducer element. The signal generator supplies the transducer(s) with a wideband electronic signal of the random or pseudo-random type.

U.S. Pat. No. 6,350,245 to Cimino, relevant portions of which are incorporated herein by reference, describes a hand-held ultrasonic surgical apparatus with a focusing lens for fragmenting or emulsifying a predetermined volume of a medium. The medium is located generally near a focal length from a concave surface of the focusing lens, and the apparatus is described to treat the medium without significant heating of the medium. The apparatus includes a housing to be held and manipulated by a surgeon or physician and an acoustic assembly mounted within the housing. The acoustic assembly has a resonant vibratory frequency that is primarily determined by the length of the acoustic assembly and an axis along which the ultrasonic vibratory energy is directed. The range for the resonant vibratory frequency to achieve sufficient focusing and sufficient ultrasonic power to fragment or emulsify tissue is between 100 kHz and 250 kHz. The acoustic assembly includes an ultrasonic motor, a rear driver, a front driver, a compression fastener, and a focusing lens.

The following patents and patent application Publications, relevant portions of which are incorporated herein by reference, may be of interest:

US Patent Application Publications 2005/0154308, 2005/0154309, 2005/0193451, 2004/0217675, 2005/0154295, 2005/0154313, 2005/0154314, 2005/0154431, 2005/0187463, 2005/0187495, 2006/0122509, 2003/0083536, 2005/0261584, 2004/0215110, 2006/0036300, 2002/0193831, and 2006/0094988, U.S. Pat. Nos. 5,143,063, 5,590,653, 6,730,034, 6,450,979, 6,607,498, 6,626,854, and 6,971,994, and PCT Patent Publications WO/2000/053263, and WO/2005/074365.

The following references, relevant portions of which are incorporated herein by reference, may be of interest:

Akashi N et al., “Acoustic properties of selected bovine tissue in the frequency range 20-200 MHz,” J Acoust Soc Am. 98(6):3035-9 (1995)

Apfel R E et al., “Gauging the likelihood of cavitation from short-pulse, low-duty cycle diagnostic ultrasound,” Ultrasound Med. Biol. 17: 179-85 (1991)

Church C C et al., “‘Stable’ inertial cavitation” Ultrasound Med Biol. 27(10):1435-7 (2001)

Fan X et al., “Control of the necrosed tissue volume during noninvasive ultrasound surgery using a 16-element phased array” Med Phys. 22(3):297-306 (1995)

Feng R et al., “Enhancement of ultrasonic cavitation yield by multi-frequency sonication” Ultrason Sonochem. 9(5):231-6 (2002)

Hakulinen U., “Potential bioeffects of diagnostic ultrasound,” Report, LUT2 pp. 1-5 (2005) (downloaded from http://venda.uku.fi/opiskelu/kurssit/LUT2/bioeffects.pdf on Oct. 12, 2007)

Laubach H J et al., “Intense focused ultrasound: evaluation of a new treatment modality for precise microcoagulation within the skin,” Dermatol Surg 34:727-734 (2008)

Miller D L et al., “Membrane damage thresholds for 1- to 10-MHz pulsed ultrasound exposure of phagocytic cells loaded with contrast agent gas bodies in vitro” Ultrasound Med Biol. 30(7):973-7 (2004)

Moran C M et al., “Ultrasonic propagation properties of excised human skin,” Ultrasound Med Biol. 21(9):1177-90 (1995)

PCT Publication WO 05/065371 to Quistgaard et al.

PCT Publication WO 05/065409 to Quistgaard et al.

PCT Publication WO 06/080012 to Kreindel

U.S. Pat. No. 4,355,643 to Laughlin et al.

U.S. Pat. No. 5,575,772 to Lennox

U.S. Pat. No. 6,350,245 to Cimino

U.S. Pat. No. 6,508,813 to Altshuler

U.S. Pat. No. 6,758,845 to Weckwerthet al.

US Patent Application Publication 2004-0039312 to Hillstead et al.

### SUMMARY OF THE INVENTION

In some embodiments of the invention, cosmetic and/or medical apparatus is provided which comprises a tissue monitoring system and a tissue treatment system. The treatment typically includes one or more of various cosmetic treatments (e.g., body contouring by lipolysis, hair removal, wrinkle and face lift, or face-localized molding of adipose tissue). Typically, the monitoring and treatment occur in alternation, until the monitoring system determines that the treatment has been completed. For some applications, the treatment and monitoring systems are coupled to a housing, further comprising one or more, e.g., a plurality of, acoustic elements, e.g., ultrasound transducers and/or acoustic reflectors, configured to transmit high intensity energy waves in order to induce an implosion wave in tissue of a target region of the subject. The acoustic elements are typically in communication with a control unit configured to effect transmission protocols by actuating the acoustic elements to transmit various forms and/or patterns of ultrasound energy to the tissue. Typically, the acoustic elements are actuated such that implosion waves are generated while substantially avoiding cavitation within the treatment area of the tissue. The induced implosion waves are inwardly-directed, and thus move inwardly toward the treatment area. In some embodiments, the implosion wave is cylindrical, spherical, circular, or partially circular, e.g., “C”-shaped.

The housing is designed such that tissue (e.g., skin and underlying tissue) of the subject is sucked at least partially into the housing, to allow the system to monitor or treat (as appropriate) the tissue that has been sucked into the housing. In this case, the system typically transmits ultrasound energy that is designated to remain in large part within the housing and tissue therein, and generally not to affect tissue outside of the housing.

In some embodiments of the present invention, at least one (e.g., a plurality) of the plurality of acoustic elements is designated to receive and/or reflect energy. The acoustic element configured to receive energy comprise transducers which convert the energy into information capable of being processed by a processor typically located remotely from the acoustic elements, enabling reflected, scattered, or through-transmitted energy to be analyzed.

In some embodiments, the plurality of acoustic elements comprises a first ultrasound transducer which transmits treatment energy toward a second acoustic element which receives and/or reflects the energy. The received energy is transmitted to the processing unit for monitoring the treatment.

In some embodiments, apparatus for treatment and monitoring tissue comprises a single focused ultrasound transducer comprising a phased array and at least one acoustic reflector e.g., a shaped; or curved, acoustic reflector. In such an embodiment, treatment energy is transmitted from the single ultrasound transducer toward the reflector, and is received by the single ultrasound transducer. The received energy is then transmitted to the processor for monitoring. In some embodiments, the acoustic elements comprise an array of ultrasound transducers and at least one acoustic reflector e.g., a shaped, or curved, acoustic reflector. In such an embodiment, treatment energy is transmitted from at least a portion of the ultrasound transducers toward the reflector, and is received by at least a portion of transducers of the array of ultrasound transducers. The received energy is then transmitted to the processor for monitoring.

In some embodiments, the plurality of acoustic elements comprises respective first and second arrays of acoustic elements. In such an embodiment, the first and second arrays of acoustic elements are disposed in a given relationship with respect to the housing in which:

(a) at least a first one of the acoustic elements in the first array is disposed opposite at least a second one of the acoustic elements in the second array, and

(b) a first portion of the first array of acoustic elements comprises ultrasound transducers configured to transmit the energy toward at least one focus zone within a plane defined by the housing.

In some embodiments, the one or more acoustic elements are disposed with respect to the housing so as to define a portion of at least one or more shapes selected from the group consisting of: a ring, a sphere, and an ellipse.

In some embodiments, the first and second arrays comprise first and second arrays of ultrasound transducers. In such an embodiment, treatment energy is transmitted from the first array toward the second array, and is received by at least a portion of transducers of the second array, and vice versa. In some embodiments, one of the arrays comprises a plurality of acoustic reflectors. In such an embodiment, treatment energy is transmitted from the ultrasound transducers toward the reflectors, and is received by at least a portion of transducers of the array of ultrasound transducers. The received energy is then transmitted to the processor for monitoring.

Cycles of treatment and monitoring occur in a generally closed-loop manner and are repeated using different signaling parameters, until a sufficient amount of data is collected. Maps of acoustic properties or images of the tissue are reconstructed and assessed.

In some embodiments, the acoustic elements generate a series of strong positive-pressure pulses of implosion waves, to generate an implosion effect. The implosion waves are typically transmitted to the treatment area at a pulse repetition frequency of up to several kHz. In response to the transmission, an increased level of pulsatile pressure is generated at a central location within the adipose tissue. Such increased pressure is configured to implode the adipose cell at the central location thereof.

In some embodiments of the present invention, the acoustic elements generate a series of strong negative-pressure pulses of implosion waves which are directed toward the treatment area at a transmission rate of several kHz. Such strong negative-pressure pulses effect radial stretching and tearing of the adipose cell. In such an embodiment, implosion, thermal ablation, as well as some localized cavitation may occur in the treatment area. In order to reduce a level of cavitation, a rapid series of high-frequency pulses, typically at a central frequency greater than 1 MHz, are transmitted during a single treatment. In some embodiments, a series of positive-pressure pulses of implosion waves are applied to the treatment area in succession with negative-pressure pulses. The application of the positive-pressure pulses mitigates and counters the cavitation effect generated by the negative-pressure pulses. In such an embodiment, a plurality of series of negative-pressure pulses are interspersed by series of positive-pressure pulses.

In some embodiments of the present invention, a continuous wave of alternating positive-pressure and negative-pressure pulses of implosion waves is applied to the treatment area. In response to the continuous wave, increased temperature is generated at the central location of the adipose cell. Such combined effect of continuous negative and positive pressure is configured to destroy the cell at the central location.

In some embodiments, thermal ablation of adipose tissue within the treatment area is accomplished using implosion ultrasound waves. In such an embodiment, cavitation is typically avoided by increasing the frequency and decreasing the wavelength of the ultrasound waves.

In some embodiments of the present invention, the implosion waves generate an implosion effect in addition to thermal ablation in the treatment area.

In some embodiments of the present invention, the treatment system is configured to generate implosion waves such that the implosion waves induce cavitation within the treatment area.

In some embodiments of the present invention, the treatment system is configured to generate implosion waves such that the implosion waves induce thermal ablation within the treatment area.

In some embodiments of the present invention, the treatment system is configured to generate implosion waves such that the implosion waves induce both cavitation and thermal ablation within the treatment area.

In some embodiments, the treatment system may be used in combination with a monitoring system. In such an embodiment, the monitoring system is configured to assess a state of tissue of the subject, and the treatment system is configured to apply a treatment to the tissue in response to the monitoring. Typically, the monitoring and treatment occur in alternation, until the monitoring system determines that the treatment has been completed.

In some embodiments of the present invention, the housing is configured to suck the tissue of the subject at least partially into the housing, to allow the system to treat the tissue that has been sucked into the housing. In this case, the system typically transmits ultrasound energy that is designated to remain in large part within the housing and tissue therein, and generally not to affect tissue outside of the housing.

In an embodiment, the housing comprises a plurality of acoustic elements, e.g., ultrasound transducers, arranged in a circle (or other typically but not necessarily closed shape). The transducers are positioned such that ultrasound energy transmitted by the transducers remains generally within a plane defined by the circle. Similarly, in embodiments in which the monitoring system comprises the housing, the transducers are typically disposed such that they are optimized to receive ultrasound energy coming generally from within the plane.

Treatments using the treatment system may include, as appropriate, applying implosion waves, causing cell implosion, heating, tissue damage, thermal ablation, acoustic streaming, mechanical irritation, cell structure alteration, augmented diffusion, and/or a cavitation effect.

Typically, the treatment system comprises circuitry for configuring the applied energy as high intensity focused ultrasound (HIFU), using techniques known in the art.

In some embodiments, the housing comprises two generally-parallel support structures, e.g., cylinders, spaced at a predetermined distance from one another so as to define a plane between the support structures, and a support element connected to both support structures. For some applications, an electromechanical system is configured to vary the distance between the support structures. For embodiments in which the support structures comprise cylinders, the electromechanical system rotates and/or counter-rotates the cylinders after the housing comes in contact with skin of the subject. Consequently, the tissue is pinched and drawn at least partially into the plane to be subsequently monitored or treated (as appropriate) by the acoustic elements.

For some applications, the housing is flexible, e.g., to allow the treatment of limbs or other curved body parts. Alternatively, the housing is generally rigid.

For some applications, the housing comprises a flexible cuff configured to surround a limb of the subject designated for treatment. The subsets of acoustic elements are typically arranged around the cuff on a circle defined by the cuff. For some applications, the acoustic elements are configured to remain fixed at their respective locations with respect to the cuff, while the cuff moves about the limb. For other applications, an electromechanical system moves at least a portion of the acoustic elements to different locations on the cuff. In embodiments in which a monitoring system is used in combination with the treatment system, the monitoring system generally continuously generates acoustic maps or images, depicting changes occurring during a treatment of the tissue within the housing. For some applications, this allows an operator of the treatment system to locate tissue to be treated, to monitor the progress of a treatment, and to alter a parameter of the treatment in response thereto. Such a parameter may include, for example, a location of a focus of the HIFU, a positioning of the housing on the subject's skin, or a strength of the applied energy. Alternatively or additionally, the treatment system and monitoring system operate in a closed loop fashion, whereby an output of the monitoring system (e.g., a location of fatty tissue) is used as an input parameter to the treatment system, such that the treatment system can adjust its operating parameters in response to the output of the monitoring system (and, for example, heat the fatty tissue).

In an embodiment, the apparatus comprises a tracking system comprising reference sensors configured to track progress of treatments conducted on different days, or during the same procedure, by registering and recording the spatial location of the treated tissue. Typically, the spatial localization is achieved in comparison to corresponding predefined anatomical locations of the subject with respect to the housing. Alternatively, the spatial localization corresponds to coordinates in a room with respect to the housing.

In some embodiments, the devices, treatments, and monitoring techniques described herein are used in order to treat varicose veins. The transducers effecting treatment of varicose veins (and other treatments described herein) operate in a closed loop manner in which the monitoring of the treatment will automatically effect a change in parameters (e.g., energy intensity or duration of pulses) of the therapy/treatment mode in the absence of intervention by an operator.

There is therefore provided, in accordance with an embodiment of the present invention, apparatus, including:

a housing configured for placement on skin of a subject, and to draw at least a portion of the skin and underlying tissue within at least a part of the housing, the housing including:

at least first and second support structures configured to be placed in contact with a surface of skin surrounding the portion of the skin, the first support structure having a first concave surface and the second support structure having a second concave surface that faces the first concave surface;

one or more first ultrasound transducers coupled to the first support structure;

one or more second ultrasound transducers coupled to the second support structure; and

a control unit coupled to the first and second ultrasound transducers and configured to drive the first and second ultrasound transducers to induce an implosion wave in a target region under the portion of the skin.

In an embodiment, the control unit is configured to drive the first and second ultrasound transducers to induce the implosion wave in adipose tissue of the subject.

In an embodiment, the control unit is configured to drive the first and second ultrasound transducers to induce the implosion wave in soft tissue of the subject.

In an embodiment, the control unit is configured to drive the first and second ultrasound transducers to induce cavitation as a result of the implosion wave induced in the target region.

In an embodiment, the control unit is configured to drive the first and second ultrasound transducers to induce the implosion wave in the target region by generating a continuous wave of acoustic energy.

In an embodiment, the control unit is configured to drive the first and second ultrasound transducers to induce the implosion wave while substantially inhibiting cavitation within the skin and underlying tissue.

In an embodiment, control unit is configured to drive the first and second ultrasound transducers to configure the implosion wave to effect implosion and thermal ablation within the target region.

In an embodiment, the control unit is configured to drive the first and second ultrasound transducers to configure the implosion wave to effect thermal ablation and cavitation within the target region.

In an embodiment, the control unit is configured to drive the first and second ultrasound transducers to induce a series of positive-pressure implosion waves in the target region.

In an embodiment, the control unit is configured to drive the first and second ultrasound transducers to induce a series of negative-pressure implosion waves in the target region.

In an embodiment, the apparatus further includes a source of suction configured to draw the skin and the underlying tissue into the housing, and the first and second ultrasound transducers are disposed with respect to the housing so as to direct the implosion wave to the tissue within the housing.

In an embodiment, the housing includes a cuff configured to surround a limb of the subject, and the first and second ultrasound transducers are coupled to the cuff.

In an embodiment, the apparatus further includes a processing unit configured to induce a computed tomography image of the target region.

In an embodiment, the first and second support structures are each shaped to define partial ellipsoids.

In an embodiment, the first and second support structures are each shaped to define partial spheres.

In an embodiment, the first concave surface is coupled to at least one acoustic reflector, and the second concave surface is coupled to at least one acoustic reflector.

In an embodiment, the apparatus further includes at least one acoustic reflector configured to reflect transmitted energy from the one or more first ultrasound transducers and toward the target region in the tissue.

In an embodiment, the apparatus further includes a processing unit, and a portion of the one or more first ultrasound transducers is configured to receive through-transmitted energy and to transmit the through-transmitted energy to the processing unit, and the processing unit is configured to monitor a parameter of the underlying tissue in response to the through-transmitted energy transmitted to the processing unit.

In an embodiment, the first ultrasound transducers are configured to transmit a shock wave into the tissue.

In an embodiment, the first and second ultrasound transducers are configured to transmit a shock wave into the tissue.

In an embodiment, at least one of the support structures is movable with respect to the other support structure after the housing comes in contact with the skin.

In an embodiment, the housing is configured to pinch the portion of the skin and underlying tissue within the housing.

In an embodiment, the first and second ultrasound transducers include phased array transducers.

In an embodiment, the phased array transducers are configured to steer a focal zone of energy transmitted toward the target region.

In an embodiment, the control unit is configured to drive the first and second ultrasound transducers to induce a series of negative-pressure implosion waves in the target region, followed by a series of positive-pressure implosion waves.

In an embodiment, the control unit is configured to substantially inhibit cavitation within the tissue by driving the first and second ultrasound transducers to induce the series of positive-pressure implosion waves in the target region.

In an embodiment, the control unit is configured to drive the first and second ultrasound transducers to configure the implosion wave to have a frequency of between 1 and 10 MHz.

In an embodiment, the control unit is configured to drive the first and second ultrasound transducers to configure the implosion wave to have a frequency of between 2 and 5 MHz.

In an embodiment, the control unit is configured to drive the ultrasound transducers to induce in the target region a series of pulses alternating between single negative-pressure pulses and single positive-pressure pulses.

In an embodiment, the control unit is configured to drive the first and second ultrasound transducers to configure the implosion wave to have a wave pressure amplitude of between 1 MPa and 100 MPa.

In an embodiment, the control unit is configured to drive the first and second ultrasound transducers to configure the implosion wave to have a wave pressure amplitude of between 10 MPa and 100 MPa.

In an embodiment, the control unit is configured to drive the first and second ultrasound transducers to induce a series of pulses of positive-pressure implosion waves in the target region.

In an embodiment, the control unit is configured to drive the first and second ultrasound transducers to configure the series of pulses to have a pulse repetition frequency of between 0.5 kHz and 50 kHz.

In an embodiment, the control unit is configured to drive the first and second ultrasound transducers to configure the series of pulses to have a pulse repetition frequency of between 0.5 kHz and 5 kHz.

In an embodiment, the control unit is configured to drive the first and second ultrasound transducers to apply a series of pulses of negative-pressure implosion waves to the tissue of the subject.

In an embodiment, the control unit is configured to drive the first and second ultrasound transducers to configure the series of pulses to have a pulse repetition frequency of between 0.5 kHz and 50 kHz.

In an embodiment, the control unit is configured to drive the first and second ultrasound transducers to configure the series of pulses to have a pulse repetition frequency of between 0.5 kHz and 5 kHz.

In an embodiment, the control unit is configured to drive the first and second ultrasound transducers to induce a series of pulses of negative-pressure implosion waves followed by a series of pulses of positive-pressure implosion waves in the target region.

In an embodiment, the control unit is configured to drive the first and second ultrasound transducers to induce a series of pulses having a pulse repetition frequency of between 0.5 kHz and 50 kHz.

In an embodiment, the control unit is configured to drive the first and second ultrasound transducers to induce a series of pulses having a pulse repetition frequency of between 0.5 kHz and 5 kHz.

In an embodiment, the underlying tissue includes adipose tissue and non-adipose tissue, and the control unit is configured to drive the first and second ultrasound transducers to selectively apply the implosion wave to the adipose tissue and to damage the adipose tissue.

In an embodiment, the control unit is configured to drive the first and second ultrasound transducers to selectively apply the implosion wave to the adipose tissue and to effect thermal ablation of the adipose tissue.

In an embodiment, the control unit is configured to drive the first and second ultrasound transducers to selectively apply the implosion wave to the adipose tissue and to effect pressure-based damage of the adipose tissue.

In an embodiment, the control unit is configured to drive the first and second ultrasound transducers to effect thermal ablation of the adipose tissue while substantially inhibiting cavitation within the adipose tissue.

In an embodiment, the control unit is configured to drive the first and second ultrasound transducers to configure the implosion wave to effect implosion and thermal ablation of the adipose tissue.

There is additionally provided, in accordance with an embodiment of the present invention, apparatus, including:

a housing configured for placement on skin of a subject, and to draw at least a portion of the skin and underlying tissue within at least a part of the housing, the housing including:
at least first and second support structures configured to be placed in contact with a surface of skin surrounding the portion of the skin, the first support structure having a first concave surface and the second support structure having a second concave surface that faces the first concave surface;
two or more first electrodes coupled to the first support structure;
two or more second electrodes coupled to the second support structure; and

a control unit coupled to the first and second electrodes and configured to drive the first and second electrodes to induce an implosion wave in a target region under the portion of the skin.

In an embodiment, the first concave surface is coupled to at least one reflector, and the second concave surface is coupled to at least one reflector.

In an embodiment, the control unit is configured to drive the two or more first electrodes to transmit a shock wave to the adipose tissue.

In an embodiment, the control unit is configured to drive the first and second electrodes to transmit a shock wave to the adipose tissue.

There is yet additionally provided, in accordance with an embodiment of the present invention, apparatus, including:

a housing configured to be coupled to a portion of skin of a subject;

one or more ultrasound transducers coupled to the housing and disposed with respect to the housing so as to be on opposing sides of the portion of the skin when the housing is coupled to the skin; and

a control unit coupled to the one or more ultrasound transducers and configured to drive the one or more ultrasound transducers to induce an implosion wave in a target region under the portion of the skin.

In an embodiment, the control unit is configured to drive the one or more ultrasound transducers to configure the implosion wave to effect implosion and cavitation within the target region.

In an embodiment, at least a portion of the one or more ultrasound transducers include a plurality of ultrasound transducers which are configured as a phased array, and the phased array of ultrasound transducers is configured to steer a focal zone of energy transmitted within the target region.

In an embodiment, the one or more ultrasound transducers are disposed with respect to the housing so as to define a portion of at least one or more shapes selected from the group consisting of: a ring, a sphere, an ellipsoid, and an ellipse.

In an embodiment, the apparatus includes at least one acoustic reflector configured to reflect through-transmitted energy from the one or more ultrasound transducers and toward a focal point in the target region.

In an embodiment:

the one or more ultrasound transducers include:
a first portion of ultrasound transducers configured to transmit treatment energy toward the target region, and
a second portion of ultrasound transducers configured to receive at least a portion of through-transmitted energy from the first portion, and

the control unit includes a processing unit configured to monitor a change in a parameter of tissue underlying the skin, responsively to the received energy.

In an embodiment, in response to the monitoring, the control unit is configured to alter treatment parameters of the first portion of the ultrasound transducers.

In an embodiment, the processing unit is configured to detect adipose tissue in the target region.

In an embodiment, the processing unit is configured to differentiate between types of tissue in the target region.

In an embodiment, the processing unit is configured to generate a computed tomography (CT) image of the target region in response to the received energy.

In an embodiment, in response to the through-transmitted energy received by the second portion of ultrasound transducers, the processing unit is configured to monitor acoustic properties of tissue in the target region and generate a temperature map based on the acoustic properties of the tissue.

In an embodiment, the first portion of ultrasound transducers and the processing unit are configured to cycle repeatedly between (a) applying a treatment tissue in the target region in response to a monitored state of the target region, and (b) monitoring the state of the target region following (a).

In an embodiment, the second portion of ultrasound transducers is configured to receive scattered waves from tissue in the target region, and, in response to scattered waves received by the second portion of ultrasound transducers, the processing unit is configured to monitor acoustic properties of the tissue and generate a temperature map based on the acoustic properties of the tissue.

In an embodiment, the processing unit is configured to generate a computed tomography (CT) image of the tissue in response to the received energy.

In an embodiment, in response to the monitoring, the control unit is configured to alter treatment parameters of the first portion of the ultrasound transducers.

In an embodiment, the processing unit is configured to detect adipose tissue in the target region.

In an embodiment, the processing unit is configured to differentiate between types of tissue in the target region.

There is further provided, in accordance with an embodiment of the present invention, a method, including:

placing on a portion of skin of a subject a housing coupled to one or more ultrasound transducers that are disposed with respect to the housing so as to be on opposing sides of the portion of the skin when the housing is coupled to the skin; and

configuring the one or more ultrasound transducers to induce an implosion wave in a target region under the portion of the skin.

There is yet further provided, in accordance with an embodiment of the present invention, a method, including:

placing on skin of a subject a housing including:
at least first and second support structures configured to be placed in contact with a surface of the skin,
   the first support structure having a first concave surface, the first support structure being coupled to one or more first ultrasound transducers, and
   the second support structure having a second concave surface that faces the first concave surface, the second support structure being coupled to one or more second ultrasound transducers;

drawing at least a portion of the skin and underlying tissue between the first and second support structures; and

driving the first and second ultrasound transducers to induce an implosion wave in a target region under the portion of the skin.

In an embodiment, the method includes:

during a first period, driving the first and second ultrasound transducers to induce at least one series of negative pressure pulses of acoustic energy in the target region;

during a second period subsequent to the first period, driving the first and second ultrasound transducers to induce at least one series of positive pressure pulses of acoustic energy in the target region; and

inducing the implosion wave in tissue of a subject while substantially inhibiting cavitation in tissue of the subject, by directing the pressure pulses to the target region during the first and second periods.

In an embodiment, driving the first and second ultrasound transducers to induce the implosion wave includes substantially restricting cavitation in the target region.

In an embodiment, the method includes:

transmitting toward the target region treatment energy from the first ultrasound transducer,

transmitting toward the target region treatment energy from the second ultrasound transducer,

receiving through-transmitted energy by at least one ultrasound transducer selected from the group consisting of the first ultrasound transducer and the second ultrasound transducer, and

monitoring a change in a parameter of tissue in the target region, responsively to the received energy.

In an embodiment, the method includes:

receiving scattered waves from the tissue in the target region by at least one ultrasound transducer selected from the group consisting of the first ultrasound transducer and the second ultrasound transducer,

monitoring acoustic properties of the tissue responsively to the receiving, and

generating a temperature map based on the monitoring of the acoustic properties of the tissue.

There is also provided, in accordance with an embodiment of the present invention, a method, including:

placing on skin of a subject a housing including:
at least first and second support structures configured to be placed in contact with a surface of the skin,
   the first support structure having a first concave surface, the first support structure being coupled to two or more first electrodes, and
   the second support structure having a second concave surface that faces the first concave surface, the second support structure being coupled to two or more second electrodes;

drawing at least a portion of the skin and underlying tissue between the first and second support structures; and

driving the first and second electrodes to induce an implosion wave in a target region under the portion of the skin.

The present invention will be more fully understood from the following detailed description of embodiments thereof, taken together with the drawings, in which:

### BRIEF DESCRIPTION OF THE DRAWINGS

FIG. 1 is a schematic illustration of ultrasound transducers, defining a portion of an ultrasound device, in accordance with an embodiment of the present invention;

FIG. 2 is a schematic illustration of apparatus comprising the ultrasound device of FIG. 1, positioned on tissue of a subject, in accordance with an embodiment of the present invention;

FIG. 3A is a schematic illustration of operation of the apparatus of FIG. 1 in a treatment mode, in accordance with an embodiment of the present invention;

FIG. 3B is a schematic illustration of operation of acoustic elements comprising ultrasound transducers and acoustic reflectors, in accordance with an embodiment of the present invention;

FIGS. 4 and 5 are graphs of transmitted pressure amplitude and time of treatment, in accordance with an embodiment of the present invention;

FIG. 6 is a schematic illustration of operation of the ultrasound apparatus in a treatment mode, in accordance with another embodiment of the present invention;

FIGS. 7A-B and 8 are graphs of transmitted pressure amplitude and time of treatment, in accordance with another embodiment of the present invention;

FIG. 9 is a graphical representation of a simulated pressure field generated by the ultrasound apparatus, in accordance with an embodiment of the present invention;

FIGS. 10A-B are graphical representation of a simulation of shifting of the focal point in the pressure field of FIG. 9, in accordance with an embodiment of the present invention;

FIG. 11 is a cross-sectional view of a portion of the apparatus shown in FIG. 2, in accordance with an embodiment of the present invention;

FIGS. 12A-B are schematic illustrations of the ultrasound treatment device, in accordance with another embodiment of the present invention;

FIG. 13 is a schematic illustration of a tissue-treatment device, in accordance with an embodiment of the present invention;

FIG. 14 is a schematic illustration of the ultrasound treatment device, in accordance with yet another embodiment of the present invention; and

FIG. 15 is a schematic illustration of a tracking system associated with the devices of FIGS. 1-14, in accordance with an embodiment of the present invention.

### DETAILED DESCRIPTION OF EMBODIMENTS

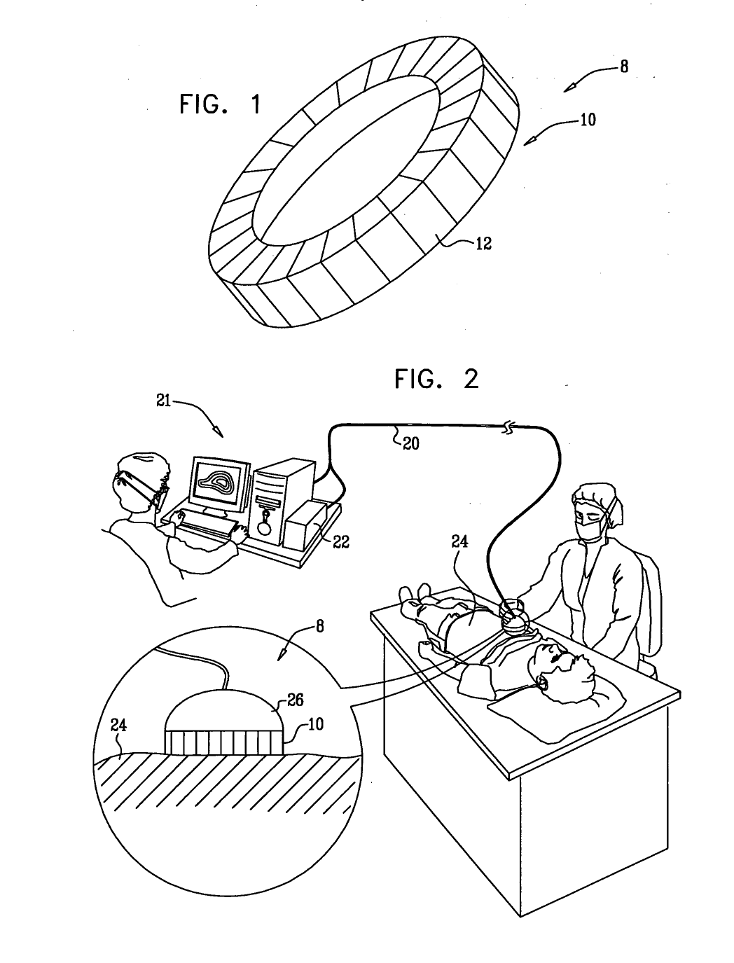

FIG. 1 is a schematic illustration of an ultrasound device 8, in accordance with an embodiment of the present invention. Ultrasound device 8 comprises a plurality of acoustic elements, e.g., ultrasound transducers 12, coupled to a support structure. The support structure maintains the transducers in a desired relationship with respect to each other, such as in a ring 10 of ultrasound transducers 12 (as shown), in another closed configuration (e.g., an ellipse), or in an open configuration (e.g., a C-shaped configuration, not shown). For some applications, the support structure comprises a rigid material, to rigidly maintain the desired relationship of the ultrasound transducers with respect to each other. For other applications, the support structure is at least somewhat flexible, to enable the ultrasound transducers to maintain suitable acoustic coupling with rounded tissue of a subject (such as a limb).

Typically, the ultrasound transducers are configured to operate as a phased array. In such an embodiment, the phased array is capable of varying its focal point in response to an electronic system which electronically reconfigures and modulates the transducers in the array. Additionally, each of the transducers may reflect through-transmitted energy back toward the respective opposing array and toward the center of the portion of the skin and underlying tissue that is drawn within a plane defined by the plurality of ultrasound transducers.

Typically, ring 10 comprises a plurality of transducers, e.g., between 8 to 32 transducers, and has a diameter of between about 20 mm and 100 mm. For example a ring having 32 transducers may have a diameter of 60 mm.

In some embodiments of the present invention, ring 10 comprises a single ultrasound transducer (configuration not shown). In such an embodiment, the transducer is curved, typically greater than 180 degrees (e.g., about 360 degrees), such that it is shaped to define ring 10. In such an embodiment, tissue is drawn within an area defined and surrounded by ring 10, and the single transducer is thus disposed on opposing sides of the tissue drawn within the area defined by the ring (configuration shown hereinbelow in FIG. 11). In such an embodiment, the single ultrasound transducer transmits a single wave which originates from opposite sides of the drawn tissue. In some embodiments, a transducer or transducers transmits as series of waves and/or pulses in a manner in which at least two waves propagate toward each other. An implosion wave is generated when the series of waves and/or pulses intersect.

FIG. 2 is a schematic illustration of ultrasound device 8, coupled to a cover 26, and positioned on skin and underlying tissue 24 of a subject, in accordance with an embodiment of the present invention. Ultrasound transducers 12 of ring 10 are typically connected via coupling lines 20 to a workstation 21 which is configured to drive and receive data from ultrasound transducers 12. Workstation 21 comprises a control unit which comprises a processing unit which processes signals from transducers 12 in order to generate acoustic maps, temperature maps, computed tomography maps, and/or images of skin and underlying tissue 24 that is enclosed within ring 10. The resultant maps or images indicate whether a desired extent of treatment has been obtained (e.g., a level of damage to tissue), and guide further treatment.

The control unit differentiates between adipose tissue and non-adipose tissue and drives ultrasound transducers 12 to selectively apply treatment energy to the adipose tissue.

It is noted that although some embodiments of the present invention are described herein with respect to generally closed-loop operation of ultrasound device 8, the scope of the present invention includes the use of ultrasound device 8 only for monitoring the tissue, while, for example, another device (e.g., a prior art ultrasound device) applies a treatment. Similarly, the scope of the present invention includes the use of ultrasound device 8 only for treating the tissue, while, for example, another device (e.g., a prior art ultrasound device) monitors the progress of the treatment. Alternatively, only monitoring is performed, or only treatment is performed.

In some embodiments, an electromechanical system 22 is typically connected to cover 26 via coupling lines 20, to generate suction under cover 26. Optionally, electromechanical system 22 dispenses ultrasound gel to enhance acoustic coupling with the tissue. Alternatively or additionally, electromechanical system 22 dispenses water for cooling the device or tissue. Further alternatively or additionally, cover 26, an inner portion of ring 10, or another component comprises a reservoir (not shown) of water and/or gel, for dispensing by an operator during a procedure.

Once the skin and underlying tissue 24 has been drawn into cover 26, low intensity ultrasound energy used for detecting a parameter of tissue 24, e.g., fat content, is transmitted between first ultrasound transducers 12. A first portion of ultrasound transducers 12 transmits energy to be received, at least in part, by a second portion of ultrasound transducers 12. Ring 10 of ultrasound transducers 12 is arranged such that the energy is transmitted from the first portion of ultrasound transducers 12, through tissue 24, e.g., typically parallel to the surface of skin of the subject, and received by the second portion of ultrasound transducers 12.

Each transducer 12 is configured to transmit treatment energy through the skin and underlying tissue 24 and receive a portion of the through-transmitted energy that is transmitted and/or reflected from other ultrasound transducers 12 of ring 10. The transducers that receive the energy convert the energy into information capable of being processed by a processor typically located remotely from the acoustic elements, enabling reflected, scattered, or through-transmitted energy to be analyzed.

The received energy is then transmitted to the processing unit for digitization and analysis. In some embodiments of the present invention, prior to, during, and following treatment of the skin and underlying tissue 24, maps of acoustic properties or images of the circular tissue area are reconstructed from energy that is received by transducers 12, typically using algorithms that are known in the art. As appropriate, the maps or images may depict various acoustic properties of the tissue, such as reflectivity, speed of sound, attenuation, acoustic impedance, and other properties. For some applications, the maps or images thus acquired are saved for later use as a reference set. In an embodiment, maps of acoustic properties are translated into maps that show tissue type within ring 10, and, for example, differentiate between fat tissue and muscle, nerve, or blood vessel cell tissues. In some embodiments, the processing unit generates a computed tomography (CT) image of the tissue in response to the energy received by the acoustic element. In some embodiments, maps of acoustic properties are translated into temperature maps, e.g., using techniques described in the above-cited PCT Publication WO 06/018837 to Azhari et al., which is incorporated herein by reference, and/or using other techniques known in the art. Further alternatively or additionally, maps of acoustic properties are assessed by computer or by a human to determine the efficacy of the treatment, and are saved or used to modify further treatments.

In some embodiments, an external source of energy is used to treat tissue 24 within ring 10. In such an embodiment, ultrasound device 8 typically works only in the monitoring mode. Maps or images are typically acquired generally continuously during the treatment. The changes derived from the treatment result in changes of the detected acoustic properties of the treated tissue. By subtracting the new maps or images from the reference set of maps or images, the amount and location of damage is assessed. Alternatively, the reference set is not used, but instead a desired endpoint is designated, and a signal is generated when the endpoint is approached or attained.

In accordance with an embodiment of the present invention, ring 10 is switched to a treatment mode, typically a plurality of times in alternation with the monitoring mode described hereinabove. In the treatment mode, ultrasound transducers 12 transmit high intensity ultrasound waves, implosion waves, shock waves, sharp negative-pressure pulses, sharp positive-pressure pulses, continuous waves (CW), pulse sequences of implosion waves, any other form of acoustical radiation that affects the tissue in a desired manner, or any combination of the above. Typically, but not necessarily, the ultrasound transducers transmit the energy in a HIFU mode.

Implosion waves are induced in response to the transmission of one or more high energy waves from one or more transducers, the one or more waves originating from opposite sides of the skin that is drawn within cover 26. For embodiments in which implosion waves are induced in response to the transmission of one wave from a single transducer, the single transducer is curved. In some embodiments, implosion waves are induced in response to the transmission of two waves originating from opposite sides of the skin that is drawn within cover 26. The two waves propagate toward each other and toward a point in the treatment zone where the wave fronts collide and generate pressure (positive or negative) having a high amplitude, e.g., between 1 and 100 MPa, typically between 10 and 100 MPa. Implosion waves are characterized by having one or more high intensity wave fronts which propagate toward the center of a zone designated for treatment. At the center, extremely high pressure and high temperatures are generated. The implosion wave induces a generally symmetrical collapse of the biological structure (e.g., tissue or cells) at its center.

For either embodiment in which one or more implosion waves propagate toward the center of the tissue treatment zone, the energy density of the one or more waves increases as the one or more waves approach the center of the tissue treatment zone.

The treatments described herein depend on the intensity of the acoustic beam, the duration of exposure, the frequency of the transmitted waves, their pulse shape and the properties of the tissue. The treatment modalities and parameters therefor described herein are configured to effect implosion of cells from their centers, thermal ablation, and/or denaturation, while substantially avoiding generation of cavitation in the treatment area. In some embodiments, the implosion waves are used to effect implosion of the cells along with cavitation.

### Thermal Ablation:

Exposing the treatment area to ultrasonic radiation typically generates localized heating in the treatment area. Typically, the heat is generated primarily in response to attenuation of the waves within the treatment medium. The amplitude of an acoustic plane wave traveling through a homogeneous tissue will decay exponentially according to:

P(x)=P₀e^{-αx}, (0.1)

where P₀ is the wave pressure at a given reference point (e.g., at the surface of the transmitting transducer), and P(x) is the wave pressure at distance x from that reference point. The constant α (alpha), or the attenuation coefficient, is a function of the characteristic of the medium through which the wave travels and a function of the ultrasound wave frequency (discussed hereinbelow).

In the following relationship of

$$I = \frac{{P}^{2}}{2 z} ,$$

the intensity of a given wave is directly proportional to the square of the pressure (P) of the wave, and is inversely proportional to twice the acoustic impedance (z). Thus, the intensity of an acoustic plane wave traveling through a homogeneous tissue will decay exponentially according to:

I(x)=I₀e^{-2αx}=I₀e^{-μx}, (0.2)

where I₀ is the wave intensity at a given reference point, and I(x) is the wave intensity at distance x from that reference point. The constant μ (mu) is simply μ=2α (alpha).

The attenuation coefficient α (alpha) depends on the wave frequency (f) and the tissue properties (a). The attenuation coefficient α (alpha) is expressed in the following equation:

α(ƒ)=a·ƒ (0.3)

When applying a wave in the lower megahertz frequency range through adipose tissue, the attenuation coefficient α (alpha) is typically 0.6 [dB/cm/MHz].

The corresponding temperature elevation ΔT resulting from the acoustic radiation at distance x from the transducer is derived (assuming a planar wave) from the following equation:

$$Δ T ( x ) = [ 2 α ( x ) \cdot I ( x ) - {Q}_{out}^{′} ( x ) ] \cdot \frac{Δ t}{C \cdot ρ} , \qquad ( 0.4 )$$

where Q′ₒᵤₜ(x) is the heat removal rate by the body per unit volume at a given distance, Δt is the time of exposure, ρ is the density (0.95 [gr/cc] for adipose tissue) and C is the specific heat constant.

In accordance with equation 0.4, it is noted that a high intensity ultrasound wave will yield a greater increase in temperature for a given duration of applying the ultrasound radiation. Additionally, it is noted that a greater attenuation coefficient (equation 0.3), will generate a higher temperature rise.

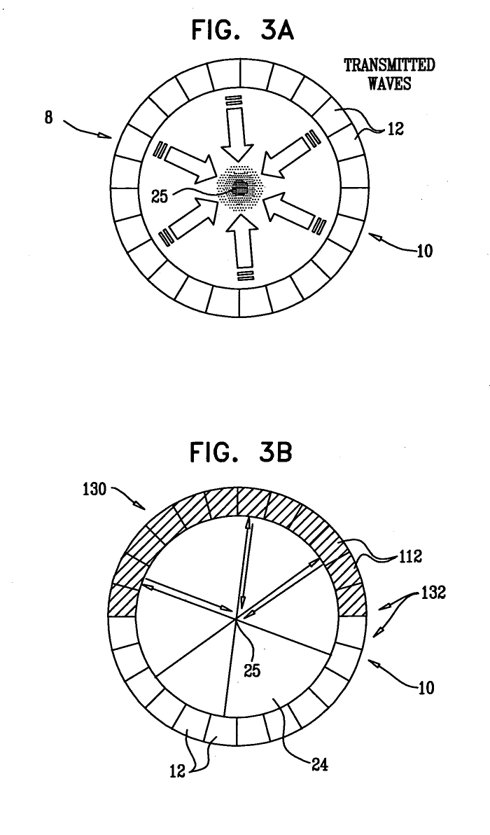

Reference is now made to FIG. 3A, which is a schematic illustration of the operation of ultrasound device 8, in accordance with an embodiment of the present invention. During the treatment mode, some or all of ultrasound transducers 12 transmit, simultaneously or in a temporal pattern, one or more high intensity waves which originate from opposite sides, of the skin toward a target region under the portion of the skin. The target region constitutes a treatment focus zone 25 in the central zone of ring 10. The wave or waves which propagate toward the center from multiple directions constitutes the implosion wave. If the pressure at the peak point of the implosion wave is positive, then a crushing force is applied to the tissue. If the pressure at the peak point of the implosion wave is negative, then a tearing force is applied to the tissue, and in some cases, cavitation is generated as a result of the implosion wave. (It is noted that the implosion wave is generated regardless of the presence or absence of cavitation, and that the use of the term “implosion wave” in this context does not refer to a secondary effect due to the collapse of bubbles generated by cavitation, but instead refers to the converging wave(s) arriving at the treatment focus zone.)

This implosion wave (e.g., a cylindrical implosion wave or a spherical implosion wave), has a high amplitude whose pressure (positive or negative) is very high at the center of the tissue treatment zone. Consequently, damage to the tissue occurs relatively rapidly. Alternatively, other signal protocols create other ultrasound-based effects besides an cylindrical implosion wave, which, nevertheless, produce a desired level of tissue damage.

Some of the energy transmitted from transducers 12 is through-transmitted toward and received by at least a portion of transducers 12. The received energy is transmitted to the processing unit which monitors changes in parameters of the skin and underlying tissue 24 responsively to the received energy. In some embodiments, during the treatment, one or more of transducers 12 can be switched into receiving mode and the detected signal can be used for determining the progress of the treatment (e.g., by analyzing acoustic properties of the tissue and generating maps of the acoustic properties of the tissue, or by evaluating the temperature at focal zone 25). Monitoring of the tissue in conjunction with the application of the treatment energy is accomplished when at least one ultrasound transducer transmits energy toward a second ultrasound transducer which receives a portion of the transmitted energy and may passively detect echoes from the first transducer in combination with techniques for passive beam-forming (e.g., in order to modulate the spatial sensitivity of the detecting transducer).

In some embodiments of the present invention, the effective energy application pattern from ultrasound device 8 is applied in a desired direction within ring 10 (e.g., toward the point of implosion) using phased array techniques known in the art. Thus, the treatment focal zone is electronically steerable using the phased arrays of ultrasound transducers 12.

In embodiments in which monitoring is used in conjunction with treatment, following the transmission of the energy from transducers 12, ring 10 is typically switched back to the monitoring mode and damage assessment is performed. If appropriate, another iteration of high energy transmission is performed, followed by another iteration of monitoring. The procedure is repeated until satisfactory results are obtained. At this point, device 8 is removed from the treatment area and is moved (robotically or mechanically) to a new region to be treated, optionally based on feedback from the monitoring.

It is noted that by using phased array techniques, the phase of the transmitted waves from each ultrasound transducer 12 can be controlled such that the focal point of the implosion wave is moved over a significant portion of the area within ring 10, without physically moving the device. The timing of transmission of the ultrasound wave from each ultrasonic transducer 12 is set such that wave fronts transmitted from transducers 12 arrive to the focal point with generally the same phase, creating a sharp local peak in intensity which causes thermal and mechanical damage to tissue 24.

In some embodiments, a plurality of rings 10 are utilized in order to attain desired results.

Typically, the radius of the implosion wave is reduced rapidly according to the wave propagation velocity. Consequently, the ultrasound energy intensity (equation 0.2) rapidly increases and reaches elevated values at the point of the wavefront collision. The implosion typically induces a symmetrical collapse of concentric cellular material (i.e., at the focal point of the implosion waves).

Ring 10, comprising a circular array of transducers 12 (as shown in FIG. 1), facilitates controlled implosions within the tissue by simultaneous transmission of a plurality of implosion waves. Additionally, the circular configuration of transducers 12 provides tomographic (e.g., Computerized Tomography-type (CT)) quantitative images of the tissue in communication within ring 10, providing guidance and treatment monitoring. Further additionally, the circular array of transducers 12 enables generation of sufficiently high pressures and intensities at the implosion focal point (referred to herein as the “hot spot”) in order to tear the cell membranes. In some embodiments, the implosion waves are induced such that they are directed toward the “hot spot” by phase modulation.

FIG. 3B shows the operation of an ultrasound device 130 comprising a plurality of acoustic elements 132, which comprise a plurality of ultrasound transducers 12 and a plurality of acoustic reflectors 112, in accordance with an embodiment of the present invention. The plurality of acoustic elements 132 are arranged in a ring 10 in a manner in which the plurality of ultrasound transducers 12 are disposed opposite the plurality of acoustic reflectors 112. Such a configuration is shown by way of illustration and not limitation. For example, the plurality of ultrasound transducers 12 may be disposed in alternation and interspersed with respect to the plurality of acoustic reflectors 112. As appropriate for any given application, acoustic reflectors 112 may be flat or curved.

In an embodiment, ultrasound transducers 12 transmit energy toward focal zone 25, and each reflector 112 receives at least a portion of the through-transmitted energy that has passed through tissue 24. Acoustic reflectors 112 reflect the through-transmitted energy back toward focal zone 25 in order to supplement the energy originally directed toward focal zone 25.

As described hereinabove, ultrasound transducers 12 typically are configured as a phased array capable of electronically steering focal zone 25 without having to physically move the apparatus that is placed on and/or draws therewithin the skin of the subject.

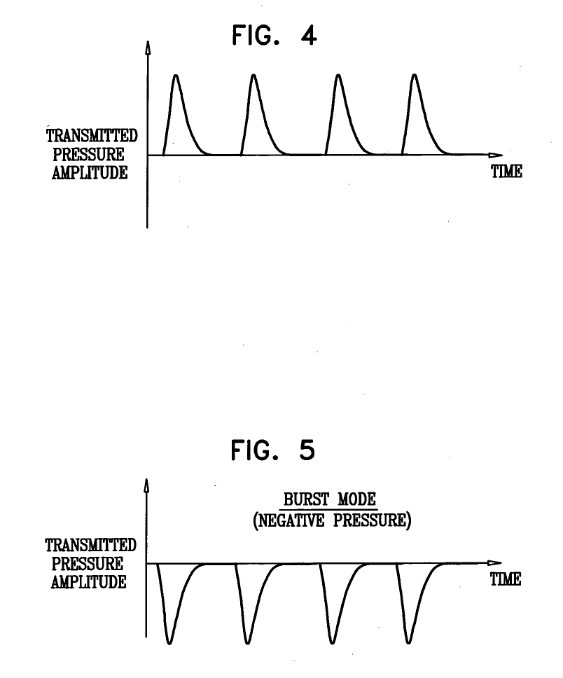

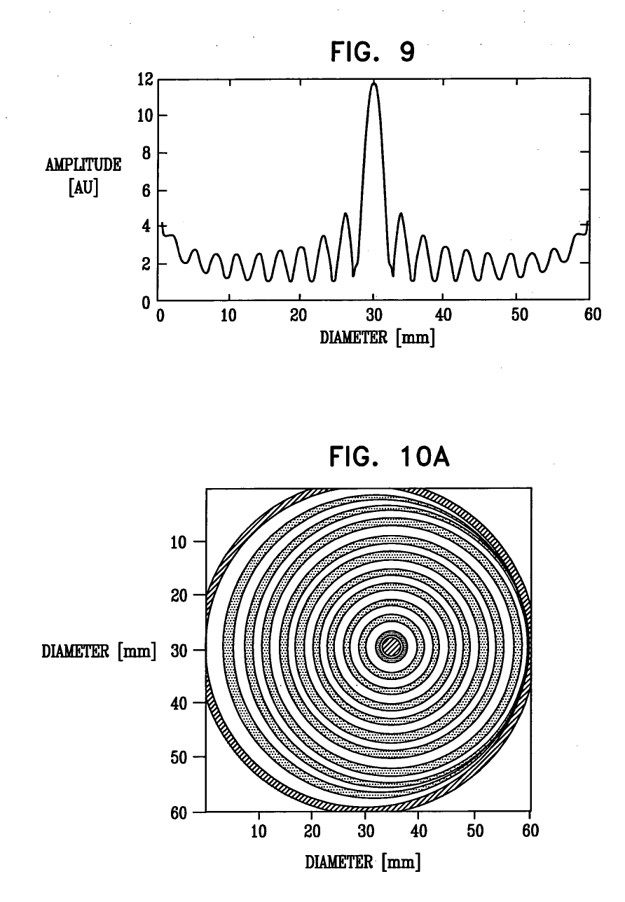

Reference is now made to FIGS. 4-5 and 7-8, which are graphs of transmitted pressure amplitude with respect to treatment time, in accordance with an embodiment of the present invention. FIGS. 4, and 7A-B, represent various transmission protocols of ultrasound energy in order to generate implosion of the cells while substantially avoiding generation of any cavitation within the treatment area.

As shown in FIG. 4, a series of strong positive-pressure pulses (PPP) of implosion waves is generated by device 8. Typically, device 8 transmits high energy waves in order to induce a series of rapid and strong pulses of implosion waves having a positive pressure in the target region of the tissue treatment zone. The pulses are typically transmitted at a pulse repetition frequency (PRF) in the kHz range (e.g., 0.5-50 kHz, typically 0.5-10 kHz). In some embodiments, a broad spectral band is transmitted, e.g., at a central frequency band of greater than 1 MHz. In other embodiments, a narrow spectral band is transmitted, e.g., at a central frequency band of less than 1 MHz. High positive pulsatile pressure is generated at the central point of the treatment area. Such an effect implodes the cellular structure from the central location, and damage of the cellular structure is effected. Some thermal effect is likely to occur. Cavitation bubbles are not likely to occur due to the application of positive pressure.

As shown in FIG. 5, a series of strong negative-pressure pulses (NPP) of implosion waves is generated by device 8. Typically, device 8 transmits high energy waves in order to induce a series of rapid and strong pulses of implosion waves having a negative pressure in the target region of the tissue treatment zone. The pulses are typically transmitted at a pulse repetition frequency in the kHz range (e.g., 0.5-50 kHz, typically 0.5-10 kHz). In some embodiments, a broad spectral band is transmitted, e.g., at a central frequency band of greater than 1 MHz. In other embodiments, a narrow spectral band is transmitted, e.g., at a central frequency band of less than 1 MHz. Low negative pulsatile pressure is generated at the central point of the treatment area. Such effect radially stretches the cells beginning from the central location of each cell. Some thermal effect is also likely occur.

By inducing the implosion waves, both radial expansion of cells in zone 25 as well as cavitation may be effected. (It is noted that the implosion wave is generated regardless of the presence or absence of cavitation, and that the use of the term “implosion wave” in this context does not refer to a secondary effect due to the collapse of bubbles generated by cavitation.) In such an embodiment, the implosion waves are induced at a frequency and using suitable signal protocols such that cavitation is effected. Gases which are dissolved in body fluids diffuse into these cavities, forming small bubbles. When these bubbles collapse, extremely high pressure (e.g., pressure greater than 1000 Atm) and very high temperatures (e.g., temperature reaching 5000 K) are generated.

The probability of inducing cavitation bubbles by ultrasonic radiation is estimated using an experimentally derived parameter called the “Mechanical Index”. This index is expressed in the following function:

$$Mi = \frac{Max \{ {P}_{negative} \}}{\sqrt{f}} , \qquad ( 0.5 )$$

where Max{P_{negative}} is maximal negative pressure in units of MPa and f is the frequency in MHz.

Typically, a Mechanical Index greater than about 1.9 suggest a likely probability of formation of cavitation bubbles. Typically, when Max {P_{negative}}=1 MPa and f=4 MHz, the Mi equals 0.5. Typically, when the Mi is less than or equal to 0.5, the probability for cavitation is low.

As can be noted from equation 0.5, a strong negative pressure is needed in order to induce cavitation bubbles. Furthermore, as can be noted from the denominator of equation 0.5, the likelihood of inducing cavitation bubbles is inversely proportional to the value of the square root of the frequency. Thus, in order to create cavitation bubbles, lower frequencies are preferred. Therefore, in order to avoid the effect of cavitation bubbles within the treatment area, device 8 applies high-frequency pulses, thereby reducing the mechanical index and avoiding cavitation within the treatment area.

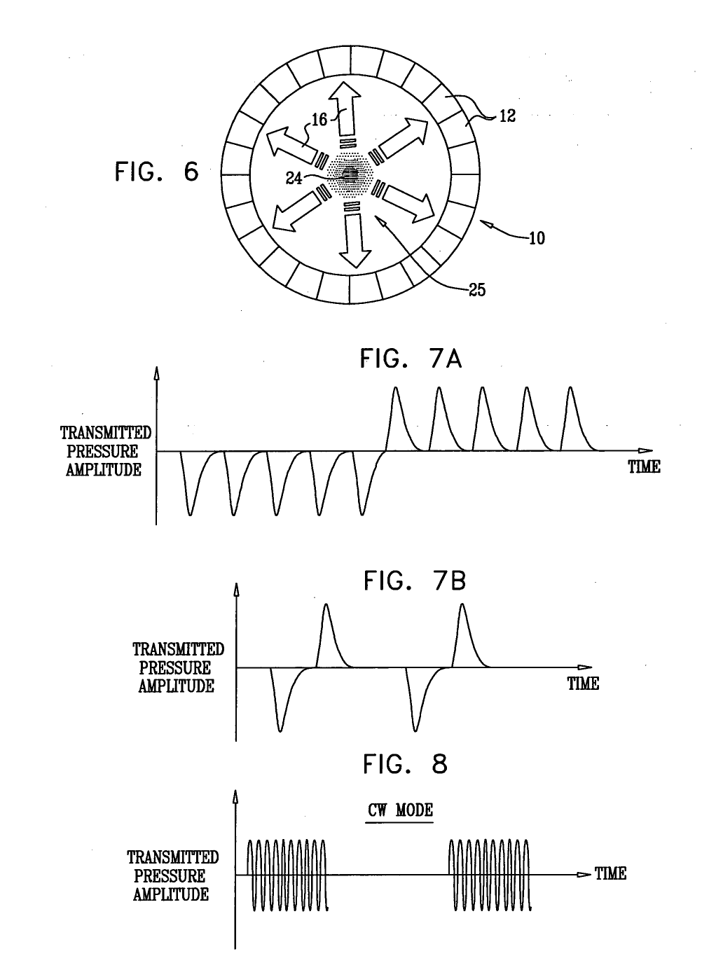

Reference is now made to FIG. 6, which is a schematic illustration of the radial expansion of tissue 24 at the implosion central point in response to the treatment described with reference to FIG. 5, in accordance with an embodiment of the present invention. Following application of the series of negative-pressure pulses from transducers 10 to the treatment zone 25, tissue 24 within the treatment zone is stretched radially (the direction of the radial stretching is indicated by arrows 16). As a result, tissue 24 and/or connective tissue are subjected to tearing stresses causing irreversible damage thereto in response to the radial stretching.

Reference is again made to FIG. 7A. A series of strong negative-pressure pulses (NPP) of implosion waves followed by a series of positive-pressure pulses of implosion waves (i.e., alternating pressure pulses) are induced in the target region of the tissue treatment zone by the transmission of high intensity waves from transducers 12 of device 8. Typically, during a first period, device 8 transmits high intensity waves which induce a series of rapid and strong pulses of implosion waves having a negative pressure in the target region of the tissue treatment zone. These negative-pressure pulses are typically transmitted at a pulse repetition frequency of (PRF) in the kHz range (e.g., 0.5-50 kHz, typically 0.5-10 kHz). In some embodiments, a broad spectral band is transmitted, e.g., at a central frequency band of greater than 1 MHz. In other embodiments, a narrow spectral band is transmitted, e.g., at a central frequency band of less than 1 MHz. Low negative pulsatile pressure is generated at the central point of the implosion of the treatment area. Such effect radially stretches the cells beginning from the central location of each cell. Furthermore, some thermal effect is also likely to occur. During a second period following the first period, device 8 transmits high intensity waves in order to induce a series of rapid and strong, high-frequency positive-pressure pulses of implosion waves in the target region of the tissue treatment zone. These positive-pressure pulses are typically transmitted at a pulse repetition frequency in the kHz range (e.g., 0.5-50 kHz, typically 0.5-10 kHz). In some embodiments, a broad spectral band is transmitted, e.g., at a central frequency band of greater than 1 MHz. In other embodiments, a narrow spectral band is transmitted, e.g., at a central frequency band of less than 1 MHz. The effects of the positive-pressure waves mitigate and counter any cavitation which may occur in response to the negative-pressure pulses of implosion waves.

It is to be noted that device 8 may cyclically transmit the series of negative and positive-pressure pulses. For example, the series depicted in FIG. 7 may be repeated until a desired level of treatment is achieved.

Reference is again made to FIG. 7B. A series of alternating pressure pulses, e.g., a strong negative-pressure pulse (NPP) of an implosion wave followed by a positive-pressure pulse (PPP) of implosion waves, is induced in the target region of the tissue treatment zone in response to the transmission of high intensity waves by transducers 12 toward the treatment area. These pulses are typically transmitted at a pulse repetition frequency of (PRF) in the kHz range (e.g., 0.5-50 kHz, typically 0.5-10 kHz). In some embodiments, a broad spectral band is transmitted, e.g., at a central frequency band of greater than 1 MHz. In other embodiments, a narrow spectral band is transmitted, e.g., at a central frequency band of less than 1 MHz. The effect of a positive-pressure pulse immediately following a negative-pressure pulse mitigates and counters any cavitation which may occur in response to the negative-pressure pulse of the implosion waves.

Reference is again made to FIG. 8. Transmission is performed by transducers 12 in a continuous wave (CW) mode, where a relatively long train of a sinusoidal wave is transmitted. In some embodiments, heating and cell implosion are effected as a result of the treatment procedure. For this particular application, the acoustic elements transmit ultrasound energy at a high frequency range of about 1-5 MHz, e.g., 3 MHz) in the CW mode. Typically, the wave has a small wavelength W, e.g., 0.5 mm. Such a wavelength is suitable for fine cosmetic treatment. Such transmission heats the treatment area to a relatively-high temperature of about 40-70 C, e.g., 45 C. Thus applying the continuous wave typically induces implosion of the cell and/or thermal ablation of the cell. In an embodiment, the temperature is evaluated using techniques described in PCT Publication WO 06/018837 to Azhari, which is incorporated herein by reference.

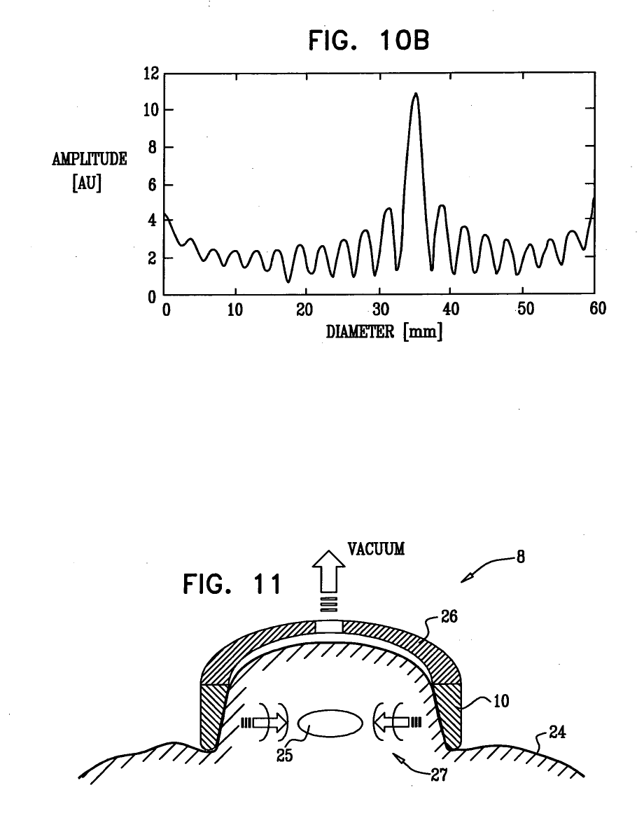

FIG. 9 is a graph of a simulated pressure field generated at the implosion point of a treatment area in response to techniques described hereinabove with reference to FIGS. 1-8, in accordance with an embodiment of the present invention. The simulated data shown represent data obtainable using 16 acoustic elements that are ultrasound transducers and are arranged in a ring having a diameter of 60 mm. A sharp pressure peak (at approximately 12 arbitrary units of amplitude) is formed at a central location of the treatment area, i.e., 30 mm, as shown. Such pressure generated by the implosion waves/and or pulses thereof is configured to damage the cells, typically by implosion. It is to be noted that when transmitting a negative-pressure implosion wave, the sign of this peak will be inverted.

FIGS. 10A-B are graphical representations of a simulation of shifting of the focal point in the pressure field as described hereinabove with reference to FIG. 9, in accordance with an embodiment of the present invention. The graph of FIG. 10A shows a shift of the focal point 5 mm to the right of the center of the treatment area defined by the ring of acoustic elements. The graph of FIG. 10B shows a sharp pressure peak (at approximately 11 arbitrary units of amplitude) at 35 mm. As described hereinabove, the ultrasound transducers are configured as a phased array which enables steering/shifting of the focal zone without physically moving the apparatus that is coupled to the skin. It is to be noted that although FIGS. 10A-B represent a shift of the focal zone of 5 mm, the focal zone may be effectively shifted 15 mm, or any other arbitrary distance, from the center of the treatment zone.

Reference is now made to FIG. 11, which is a schematic illustration of a portion of ultrasound device 8, in accordance with an embodiment of the present invention. In an embodiment, the operator (as shown) or a robotic system moves ultrasound device 8 to different sites on tissue 24. For example, the tissue may be skin overlying a significant deposit of fat, and the subject may be undergoing a cosmetic procedure to remove the fat. In some embodiments, vacuum is applied within a space defined by cover 26 in order to draw tissue 24 into ring 10. Alternatively, other techniques (such as suction or pinching by hand or by a pinching tool) are used to draw the tissue into ring 10.

Once tissue 24 is firmly secured within ring 10, good acoustic coupling between the tissue and the ring is typically verified, prior to ultrasound device 8 entering a monitoring mode, for example, by transmitting “scout” waves from one side of the ring to the other. Following the drawing of the tissue into ring 10, ultrasound waves 27 are transmitted from device 8 toward a treatment focus zone 25.

It is to be noted that the device 8 described herein with respect to a suction device transmits waves 27 by way of illustration and not limitation. Device 8 may transmit ultrasound energy in the form of waves or pulses using one of the techniques described herein with reference to FIGS. 1-9.

As described hereinabove with reference to FIG. 1, ring 10 may comprise a plurality of ultrasound transducers or a single ultrasound transducer disposed on opposing sides of the tissue.

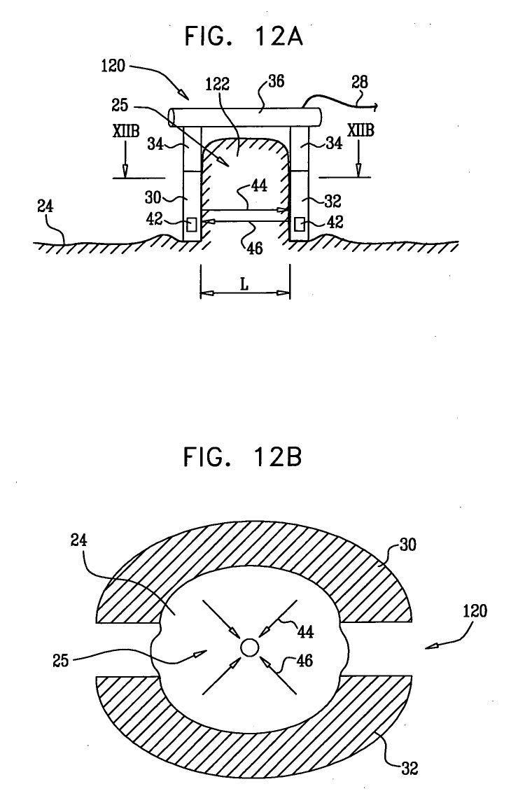

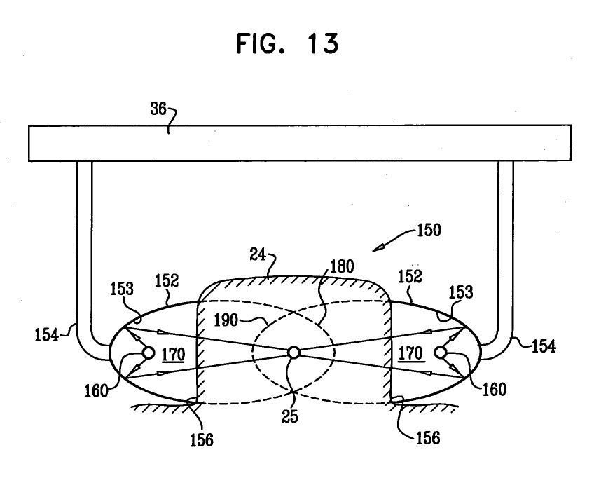

FIG. 12A is a schematic illustration of a system 120 for lipolysis and body contouring, comprising a housing 50, a plurality of acoustic elements comprising a subset 30 and a subset 32 of the acoustic elements, in accordance with an embodiment of the present invention. Each subset comprises one or more acoustic elements, e.g., ultrasound transducers and/or acoustic reflectors. At least a pair of acoustic elements are disposed at respective locations with respect to housing 50. Housing 50 is typically but not necessarily rigid, and comprises a support element 36 connected to ends of two support structures 34, e.g., cylinders, as shown, or members that are shaped in a different manner. In an embodiment, housing 50 is flexible, at least in part.

Support structures 34 are disposed (a) at an angle, e.g., generally perpendicularly, with respect to support element 36, and (b) at an angle, e.g., generally perpendicularly, with respect to a surface of skin surrounding portion 122 of the skin. For some embodiments, one or both support structures 34 extend somewhat outward, e.g., by being disposed at an angle between 90 and 160 degrees with respect to support element 36, such that the foci of transmitted waves from subsets 30 and 32 overlap within portion 122.

Subsets 30 and 32 are disposed upon support structures 34, which are spaced at a distance L from one another. Distance L typically ranges from about 5 mm to about 150 mm, e.g., about 5 mm to 40 mm or 40 mm to 150 mm. The space between support structures 34 defines a plane in which tissue 24 designated for treatment is drawn into housing 50. For some applications, an electromechanical system (not shown) is connected via lead 28 to support element 36 and moves support structures 34 in a controlled motion, varying distance L between support structures 34. When housing 50 is placed on tissue 24 designated for monitoring or treatment (as appropriate), such motion pinches and draws tissue 24 into the plane defined by housing 50 (configuration shown in FIG. 11B). Alternatively or additionally, support structures 34 rotate in the same direction or in opposite directions, to draw new tissue into the plane.

For some applications, the electromechanical system is disposed upon support structures 34. For other applications, a source of suction, e.g., a vacuum pump disposed upon housing 50 draws portion 122 of skin and underlying tissue 24 into housing 50.

Once tissue 24 has been drawn into housing 50, low intensity ultrasound energy used for detecting a parameter of portion 122 of tissue 24, e.g., fat content, is transmitted between first subset 30 and second subset 32 toward treatment focus zone 25. A first portion of first subset 30 transmits energy to be received, at least in part, by a first portion of second subset 32. Alternatively or additionally, a second portion of second subset 32 transmits energy to be received, at least in part, by a second portion of first subset 30. Support structures 34 are arranged such that the energy is transmitted through portion 122 of tissue 24, e.g., typically parallel to the skin of the subject, and received on subset 30 and/or subset 32. Typically, tissue 24 includes skin of the subject and energy is transmitted from either subset 30 and 32, through the skin. Energy is transmitted to tissue 24 using one or a combination of techniques described herein with reference to FIGS. 1-9.

The electromechanical system maintains distance L between first subset 30 and second subset 32 during the monitoring and treatment process. Acoustic elements in subset 30 may be moved away from acoustic elements in subset 32 due to the movement of support structures 34 by the electromechanical system. Alternatively or additionally, portions of the acoustic elements are moved to different locations with respect to support structure 34. The movement and distances between the portions of the acoustic elements are typically recorded by a linear encoder or by counting steps of a stepper motor. Such recording is useful in the monitoring of the body contouring process, as described hereinbelow.

In an embodiment, the electromechanical system moves housing 50 to different locations on tissue 24 of the subject, enabling the acoustic elements to detect the presence of adipose tissue at multiple locations on tissue 24 of the subject. For embodiments in which support structures 34 comprise cylinders, moving housing 50 comprises rotating the cylinders along tissue 24 while periodically counter-rotating the cylinders such that tissue 24 is rolled between the cylinders and introduced within housing 50. Alternatively or additionally, the rolling of the cylinders is configured to induce a form of peristaltic motion of tissue 24. For other applications, the electromechanical system is not used to move housing 50 along tissue 24 of the subject.

In some embodiments, subsets 30 and 32 each comprise a respective pair of acoustic elements, the pair comprising an ultrasonic transducer and an acoustic reflector. That is, subset 30 comprises a first ultrasound transducer and subset 32 comprises a first reflector that is disposed opposite the first transducer. Subset 32 comprises a second ultrasound transducer and subset 30 comprises a second reflector that is disposed opposite the second transducer. The reflecting elements are focused toward the central point of the treatment zone. In this configuration, an implosion wave is obtained during two consecutive transmissions. During a first transmission, a first set of waves is transmitted from the ultrasound transducers, reaches the reflectors, and is reflected back towards the center of the treatment zone. The second transmission from the ultrasound transducers is timed so that a second set of waves is transmitted from the ultrasound transducers in a manner in which the wavefronts of the second set of waves will collide, at the center of the treatment zone, with the wavefronts of the reflected first set of waves.

It is to be noted that although FIG. 12A shows support structures 34 that are cylindrical, support structures 34 may be shaped to define partial spheres or partial ellipsoids which cup and surround the skin and underlying tissue. In such an embodiment, the respective one or more acoustic elements of subsets 30 and 32 are disposed within a concave inner wall of support structures 34. Thus, the respective one or more acoustic elements of subsets 30 and 32 are shaped to define partial spheres or partial ellipsoids having concave surfaces. In either embodiment, the one or more acoustic elements of subset 30 are shaped to provide a concave surface that faces the concave surface of the one or more acoustic elements of subset 32. Typically, the partial ellipsoids or partial spheres are positioned on the skin of the subject. In some embodiments, each support structure comprises a source of suction in order to draw tissue within respective areas defined by the partial ellipsoids or partial spheres. Typically, one or more treatment focal zones are created during a single treatment by electronically controlling the phased arrays, without having to physically move the apparatus to a different location on skin of the subject.

Typically, upon detection of the presence of adipose tissue by the acoustic elements, acoustic elements from subsets 30 and 32, apply treatment energy to portion 122 of tissue 24. Subset 30 and subset 32 work in conjunction with each other in a generally closed-loop operation cycling repeatedly between (a) subsets 30 and 32 applying treatment to portion 122 of tissue 24 in response to the monitored state of portion 122, and (b) subset 30 and/or 32 monitoring the state of portion 122 of tissue 24 following (a). For some applications, portions of subsets 30 and 32 are activated simultaneously to induce an implosion wave in the plane. The intensity peak of such a wave is located between subsets 30 and 32, and its frequency and amplitude are suitable for treating portion 122 of tissue 24. The same or other portions of subsets 30 and 32 monitor waves transmitted through or reflected from portion 122, typically between successive treatments by subsets 30 and 32.

In some embodiments, subsets 30 and 32 are used for monitoring the treatment procedures while an external energy transmitter is used to transmit the ultrasound energy. In some embodiments, the energy source is coupled to housing 50 (configuration not shown), or, alternatively, mechanically separate from the housing.

In either embodiment in which the energy source or the acoustic elements are used to treat tissue 24, the energy source and/or acoustic elements comprise circuitry for focusing energy designated for the destruction of adipose tissue, such as acoustic energy (e.g., implosion waves, high intensity focused ultrasound, shock waves, sharp negative-pressure pulses, sharp positive-pressure pulses, or high intensity ultrasound waves), electromagnetic radiation (e.g., microwave radiation), laser energy, and/or visual or near-visual energy (e.g., infra-red). The energy source and/or acoustic elements transmit energy intense enough to cause damage to adipose tissue within portion 122.

Effects or combined effects of treatments by the energy source and/or acoustic elements may include, as appropriate (and as described hereinabove with reference to FIGS. 5-6 and 7-8), heating, tissue damage, thermal ablation, mechanical irritation, acoustic streaming, cell structure alteration, and/or augmented diffusion. It is to be noted that techniques described herein with reference to FIGS. 4 and 7A-B avoid generation of cavitation within tissue 24. In some embodiments, as described hereinabove, cavitation may be effected in combination with implosion of tissue 24 within treatment focus zone 25. For some applications, lipolysis is accomplished when the energy source and/or acoustic elements elevate the temperature of portion 122 of tissue 24 by less than 10 C, e.g., less than 5 C.

For some applications, the energy source and/or acoustic elements provide energy such that the treatment generates a combined effect of at least two of the above mentioned effects. For this application, energy is applied, inducing a different type of damage to the tissue. The sets are typically operated in a synchronized mode to enhance the tissue damaging process. Alternatively, a multipurpose array is used which is capable of producing at least two types of damage to a predefined tissue region by applying a plurality of transmissions (e.g., a sequence of transmissions or parallel transmissions). Inducing the at least two types of damage simultaneously or alternately creates synergism, accelerating the tissue damaging procedure and reducing the overall treatment time.

Energy is transmitted in conjunction with the monitoring of the treatment process by acoustic subsets 30 and 32. For some applications, in addition to monitoring the treatment procedure, the body contouring process is tracked by sensors 42. For example, sensors 42 may comprise electromagnetic sensors or optical sensors that are coupled to housing 50. The sensed information is transmitted to a processing unit. Storing the tracking information allows for improved follow-up and comparison of body contouring treatments conducted on different days or during the treatment.

For some applications, tracking the treatment process occurs in conjunction therewith. In response to an indication of fat content detected by the acoustic elements in a particular area of the body of the subject, the a pre-treatment map is generated and the physician marks the area, designating it for treatment. Housing 50 is subsequently placed on the designated area to provide treatment and monitoring thereof. Following the treatment, housing 50 is re-positioned in the designated area to enable tracking of the body contouring process by sensors 42. Sensors 42 help ensure that (1) treatment has been applied to all subsections of the designated area and/or (2) treatment has not been applied multiple times to the same subsection during a single session. Thus, for some applications, treatment locations during one session are stored to facilitate the initiation of treatments in subsequent locations other than already-treated regions.

FIG. 12B is a transverse cross-section of housing 50 described hereinabove with reference to FIG. 11A, in accordance with an embodiment of the present invention. Tissue 24 is pinched between subsets 30 and 32, such that treatment focus zone 25 is disposed between subsets 30 and 32. As shown, subsets 30 and 32 are shaped to define curved structures, e.g., partial ellipses, which are capable of generating implosion waves in zone 25. It is to be noted that although partial ellipses are shown, subsets 30 and 32 may be shaped to define partial circles.

Reference is now made to FIG. 13, which is a schematic illustration of a tissue treatment device 150 comprising two partially ellipsoidal housing structures 152 which pinch a portion of skin and underlying tissue 24 therebetween and generate an implosion wave in the portion of the skin and underlying tissue 24, in accordance with an embodiment of the present invention. Device 150 comprises a housing, e.g., a c-shaped clamp, comprising a horizontal support element (not shown) coupled to support structures 154 which are each coupled to a respective housing structure 152.

Support structures 154 are spaced apart from each other at a distance ranging from about 5 mm to about 150 mm, e.g., about 5 mm to 40 mm or 40 mm to 150 mm. At least one, e.g., both, of support structures 154 is configured to move axially with respect to the horizontal support element.

Pinching the skin and underlying tissue 24 between housing structures 152 allows contact of housing structures 152 with the portion of skin 24 in order to enhance the efficacy of the shock wave treatment (typically also using a gel, as is known in the art). Each housing structure 152 is shaped to define a partially ellipsoidal wall having an inner concave surface 153 and a substantially flat surface 156 which contacts the surface of skin 24 of the subject. Each housing structure 152 comprises a coupling medium 170 and at least one transducer 160. For each housing structure 152, concave surface 153 provides a reflective surface designed to reflect waves transmitted from transducer 160 toward focal zone 25 within the portion of skin. In some embodiments, concave surface 153 is coated with a reflective material. In some embodiments, concave surface 153 is coupled to at least one reflector. Typically, energy transmitted from each housing structure 152 is transmitted in a respective elliptical energy-transmission zone 180 and 190 in which transducers 160 are disposed at a first focus, while the treatment focal zone 25 is disposed at the second focus of each elliptical energy-transmission zone 180 and 190. As shown, the second foci of the respective elliptical energy-transmission zones 180 and 190, transmitted from each housing structure 152, overlap at treatment focal zone 25. Since two partially ellipsoidal housing structures 152 are provided which have concave surfaces 153 that face each other, an implosion wave is induced at treatment focal zone 25 when high-intensity waves propagate toward each other from either housing structure 152.

Support structures 154 are disposed (a) at an angle, e.g., generally perpendicularly, with respect to the horizontal support element, and (b) at an angle, e.g., generally perpendicularly, with respect to a surface of skin surrounding the portion of the skin pinched between housing structures 152. For some embodiments, one or both support structures 34 extend somewhat outward, e.g., by being disposed at an angle between 90 and 160 degrees with respect to support element 36, such that the foci of transmitted waves from transducers 160 in housing structures 152 overlap.

Typically, transducers 160 comprise ultrasound transducers which generate shock waves and transmit the waves toward treatment focal zone 25 in a manner as indicated by the arrows. In such an embodiment, concave inner surface 153 of each housing structure 152 comprises, at least in part, an acoustic reflective surface which reflects and focuses the acoustic waves toward treatment focal zone 25. The shock waves generated by each transducer 160 propagate towards each other in order to induce an implosion wave in the target region of the tissue.

In some embodiments, transducers 160 each comprise a pair of electrodes. When a voltage pulse is applied across the electrodes of transducers 160, an electrical discharge is generated and propagates through medium 170 in a manner as indicated by the arrows. The electrical discharge generates a shock wave in medium 170. The curved surface 153 reflects and focuses the shock wave toward treatment focal zone 25. In such an embodiment, the reflector provided by surface 153 comprises a metal reflector. It is to be noted that the reflector comprises metal by way of illustration and not limitation and that the reflector may comprise any suitable hard material, e.g., plastic.

It is to be noted that each housing structure 152 is partially ellipsoidal by way of illustration and not limitation. For example, each housing structure 152 may be partially spherical. It is to be further noted that two housing structures 152 are shown by way of illustration and not limitation. For example, three or more housing structures 152 may be coupled to the portion of skin and underlying tissue 24.

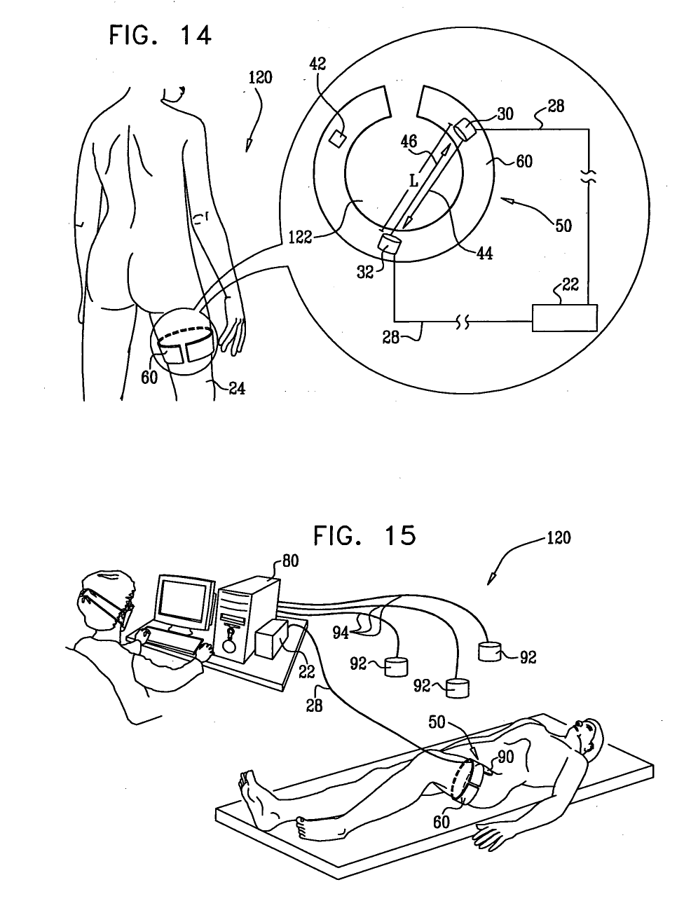

Reference is now made to FIG. 14, which is a schematic illustration of system 120 similar to the embodiments described hereinabove with reference to FIGS. 12A-B, with the exception that housing 50 comprises cuff 60. Typically, cuff 60 surrounds a limb of the subject and transmits ultrasound energy using one or a combination of treatments described hereinabove with reference to FIGS. 4, 5, 7A-B, and 8. As shown, treatment energy is transmitted from both subsets 30 and 32 in both directions (as indicated by arrows 44 and 46).

In some embodiments, cuff 60 comprises ring of transducers, as described hereinabove with reference to FIG. 1. In some embodiments, cuff 60 comprises a ring of acoustic elements comprising a plurality of ultrasound transducers and a plurality of acoustic reflectors, as described hereinabove with reference to FIG. 3B.

FIG. 15 shows system 120 comprising a tracking system comprising a plurality of reference sensors 92, in accordance with an embodiment of the present invention. Reference sensors 92 can be implemented in combination with each of the described embodiments of FIGS. 1-14, and assess the location of treated tissue 24 by registering the relative spatial coordinates of the acoustic elements and/or anatomy of the subject. The sensed information is transmitted to a processing unit 80 by leads 94 coupled to reference sensors 92. Storing the location of treated areas allows for improved follow-up and comparison of treatments conducted on different days. For some applications, location sensing is performed in conjunction with the treatment to help ensure that (1) treatment has been applied to all subsections of a designated area, and/or (2) treatment has not been applied multiple times to the same subsection during a single session. Thus, for some applications, treatment locations during one session are stored, to facilitate treatments in subsequent locations being initiated outside of already-treated regions.

Typically, housing 50 comprises a sensor 90 in communication with reference sensors 92. For some applications, reference sensors 92 are placed at predetermined locations in the treatment room. Spatial localization of housing 50 with respect to coordinates of the room is achieved when reference sensors 92 transmit signals to sensor 90 (or vice versa, or when a spatial relationship is determined between sensors 92 and sensor 90). The localization can be based on measurements using electromagnetic waves (e.g., RF-induced currents in mutually-perpendicular coils), optical information (e.g., by processing video acquired by each of sensors 92) or acoustic waves (e.g., by time-of-flight measurements). In an embodiment, sensor 90 receives signals and transmits signals back to reference sensors 92 (or vice versa). The signals are subsequently transmitted to processing unit 80. For some applications, the signals transmitted from reference sensors 92 form an electromagnetic field around the subject, capable of being sensed by sensor 90. In such an embodiment, sensor 90 communicates either actively or passively with reference sensors 92 (e.g., passive communication may utilize radio frequency identification techniques known in the art).

For some applications, reference sensors 92 are placed at predetermined locations on the body of the subject (e.g., sternum, patella, pelvis, navel, etc.), and spatial localization of the housing and treated tissue relative to the anatomical landmarks is achieved.

In some embodiments, the spatial localization procedure is initiated by an operator, e.g., using a wand comprising reference sensor 92. The operator contacts predetermined anatomical landmarks of the subject and references the coordinates thereof with respect to housing 50.

For some applications, the spatial location of housing 50 during the treatment procedure is automatically registered along with other details such as intensity and duration of each treatment stage. This information is stored in processing unit 80 and used in following sessions as a reference for monitoring the treatment process.

For some applications, the physician may choose to save the obtained mapping information in the system memory of processing unit 80. In such a case, the spatial map may be recorded and graphically presented on a suitable display device. In an embodiment, graphical overlay of the spatial map generated during the treatment procedure is superimposed upon the pre-treatment map, thus indicating the damage to the tissue effected by the treatment procedure. For some applications, when the treatment required for the tissue region has been completed, an ink or other marking is stamped on the subject's skin, and the vacuum suction is then released. When the operator moves the device to a second region on the skin, its spatial orientation relative to the previously treated region, i.e., the marked region, is typically displayed via an electronic display. Alternatively or additionally, the spatial orientation of the second region is viewed in comparison with the marked area without the use of an electronic display, thus allowing the operator to monitor the progress of the entire session during the treatment procedure.

It is to be noted that the tracking system is used in combination with cuff 60 by way of illustration and not limitation, and that the tracking system can be implemented in combination with each of the described embodiments of FIGS. 1-14.

It is noted that although some embodiments of the present invention are described with respect to the use of ultrasound, the scope of the present invention includes replacing the ultrasound transducers described herein with transducers of other forms of energy, such as electromagnetic radiation.

It is to be further noted that, in some embodiments, inducing implosion waves (generated using techniques described herein) is not limited only to cosmetic treatments. For example, the implosion waves may be induced in tissue in order to treat tumors, e.g., breast cancer tumors.

Reference is now made to FIGS. 1-15. In some embodiments, apparatus comprise circuitry for focusing energy designated for the destruction of adipose tissue, such as acoustic energy (e.g., high intensity focused ultrasound, shock waves, sharp negative pressure pulses, or high intensity ultrasound waves). It is to be noted that acoustic elements, e.g., ultrasound transducers, are described herein by way of illustration and not limitation. For example, elements other than acoustic elements may be used to induce the implosion waves, in a manner as described hereinabove with respect to techniques for inducing the implosion waves using acoustic elements. For example, apparatus described herein may comprise transducers which transmit and/or receive electromagnetic radiation (e.g., microwave radiation or radiofrequency), laser energy, and/or visual or near-visual energy (e.g., infrared).

Reference is again made to FIGS. 1-15. Apparatus described herein typically operates in a closed-loop manner in which the monitoring of the treatment automatically effects a change in parameters (e.g., energy intensity, energy target site, or duration of pulses) of the therapy/treatment mode in the absence of intervention by an operator for effecting the change. For embodiments in which the transducers comprise ultrasound transducers, during the monitoring, through-transmitted and/or scattered waves are received, and information representing acoustical properties of those waves (e.g., speed of sound, attenuation coefficient, etc.) is transmitted to the processing unit. For embodiments in which the transducers comprise electrodes, during the monitoring, through-transmitted and/or scattered waves are received and information representing properties of those waves is transmitted to the processing unit. In either embodiment, the processing unit typically generates a temperature map based on the information in order to determine the temperature at the treatment focal zone and whether to continue applying treatment energy to the zone. Additionally, the processing unit analyzes the information in order to assess an extent of tissue damage. Further additionally, the processing unit analyzes the information in order to differentiate between different tissue types, e.g., to detect adipose tissue.

Typically, in the embodiments described hereinabove, confocal acoustic radiation is achieved by the coaxial positions of the acoustic elements with respect to the housing, or by other suitable positions of the acoustic elements. In these embodiments, such confocal acoustic radiation enables the acoustic elements to transmit treatment energy toward a focal zone in the center of the portion of the skin in less time or energy flux than it would take for a single transducer to achieve a similar tissue treatment, because the focal zone is receiving confocal acoustic beams from either direction.

In an embodiment, techniques and apparatus described in one or more of the following patents and patent applications are combined with techniques and apparatus described herein:
U.S. Provisional Patent Application 60/780,772 to Azhari et al., entitled, “A method and system for lypolysis and body contouring,” filed Mar. 9, 2006;
U.S. Provisional Patent Application 60/809,577 to Azhari et al., entitled, “A device for ultrasound monitored tissue treatment,” filed May 30, 2006;
U.S. Provisional Patent Application 60/860,635 to Azhari et al., entitled, “Cosmetic tissue treatment using ultrasound,” filed Nov. 22, 2006;
U.S. Regular patent application Ser. No. 11/651,198 to Azhari et al., entitled, “A device for ultrasound monitored tissue treatment,” filed Jan. 8, 2007;
U.S. Regular patent application Ser. No. 11/653,115 to Azhari et al., entitled, “A method and system for lipolysis and body contouring,” filed Jan. 12, 2007;
International Patent Application PCT/IL2007/000307 to Azhari et al., entitled, “A device for ultrasound monitored tissue treatment,” filed on Mar. 8, 2007;
U.S. Provisional Patent Application 60/999,139 to Azhari et al., entitled, “Implosion techniques for ultrasound,” filed Oct. 15, 2007; and/or
A US provisional patent application to Azhari et al., entitled, “A device for ultrasound treatment and monitoring tissue treatment,” filed Sep. 12, 2008.

For some applications, techniques described herein are practiced in combination with techniques described in one or more of the references cited in the Cross-references section or Background section of the present patent application, which are incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-18. (canceled)

19. The apparatus according to claim 55, wherein the control unit is configured to drive the one or more ultrasound transducers to transmit a shock wave into the target region.

20-45. (canceled)

46. Apparatus, comprising:
a housing configured for placement on skin of a subject, and to draw at least a portion of the skin and underlying tissue within at least a part of the housing, the housing comprising:
at least first and second support structures configured to be placed in contact with a surface of skin surrounding the portion of the skin, the first support structure having a first concave surface and the second support structure having a second concave surface that faces the first concave surface;
two or more first electrodes coupled to the first support structure;
two or more second electrodes coupled to the second support structure; and
a control unit coupled to the first and second electrodes and configured to drive the first and second electrodes to induce an implosion wave in a target region under the portion of the skin.

47-54. (canceled)

55. Apparatus, comprising:
a housing configured to be coupled to a portion of skin of a subject;
one or more ultrasound transducers coupled to the housing and disposed with respect to the housing so as to be on opposing sides of the portion of the skin when the housing is coupled to the skin; and
a control unit coupled to the one or more ultrasound transducers and configured to drive the one or more ultrasound transducers to induce an implosion wave in a target region under the portion of the skin.

56. (canceled)

57. The apparatus according to claim 55, wherein the control unit is configured to drive the one or more ultrasound transducers to induce the implosion wave in soft tissue of the subject.

58. The apparatus according to claim 55, wherein the control unit is configured to drive the one or more ultrasound transducers to induce cavitation as a result of the implosion wave induced in the target region.

59. (canceled)

60. The apparatus according to claim 55, wherein the control unit is configured to drive the one or more ultrasound transducers to induce the implosion wave while substantially inhibiting cavitation within the target region.

61-63. (canceled)

64. The apparatus according to claim 55, wherein the control unit is configured to drive the one or more ultrasound transducers to induce a series of positive-pressure implosion waves in the target region.

65. The apparatus according to claim 55, wherein the control unit is configured to drive the one or more ultrasound transducers to induce a series of negative-pressure implosion waves in the target region.

66. (canceled)

67. The apparatus according to claim 55, wherein the housing comprises a cuff configured to surround a limb of the subject, and wherein the one or more ultrasound transducers are coupled to the cuff.

68-70. (canceled)

71. The apparatus according to claim 55, wherein the housing comprises:
at least first and second support structures configured to be placed in contact with a surface of skin surrounding the portion of the skin, the first support structure having a first concave surface and the second support structure having a second concave surface that faces the first concave surface, and wherein the one or more ultrasound transducers comprise:
one or more first ultrasound transducers coupled to the first support structure, and
one or more second ultrasound transducers coupled to the second support structure.

72. The apparatus according to claim 71, wherein the first and second support structures are each shaped to define partial ellipsoids.

73. The apparatus according to claim 71, wherein the first and second support structures are each shaped to define partial spheres.

74. The apparatus according to claim 71, further comprising at least one acoustic reflector configured to reflect transmitted energy from the first and second ultrasound transducers and toward the target region in the tissue.

75-84. (canceled)

85. The apparatus according to claim 55, wherein:
the one or more ultrasound transducers comprise:
a first portion of ultrasound transducers configured to transmit treatment energy toward the target region, and
a second portion of ultrasound transducers configured to receive at least a portion of through-transmitted energy from the first portion, and
wherein the control unit comprises a processing unit configured to monitor a change in a parameter of tissue underlying the skin, responsively to the received energy.

86. The apparatus according to claim 85, wherein, in response to the monitoring, the control unit is configured to alter treatment parameters of the first portion of the ultrasound transducers.

87. The apparatus according to claim 85, wherein the processing unit is configured to detect adipose tissue in the target region.

88. (canceled)

89. The apparatus according to claim 85, wherein the processing unit is configured to generate a computed tomography (CT) image of the target region in response to the received energy.

90. The apparatus according to claim 85, wherein, in response to the through-transmitted energy received by the second portion of ultrasound transducers, the processing unit is configured to monitor acoustic properties of tissue in the target region and generate a temperature map based on the acoustic properties of the tissue.

91-96. (canceled)

97. The apparatus according to claim 85, wherein:
the processing unit is configured to differentiate between types of tissue in the target region,
the types of tissue include adipose tissue and non-adipose tissue, and
the control unit is configured to drive the first and second portions of ultrasound transducers to selectively induce the implosion wave in the adipose tissue and to damage the adipose tissue.

98-103. (canceled)

104. A method, comprising:
placing on a portion of skin of a subject a housing coupled to one or more ultrasound transducers that are disposed with respect to the housing so as to be on opposing sides of the portion of the skin when the housing is coupled to the skin; and
configuring the one or more ultrasound transducers to induce an implosion wave in a target region under the portion of the skin.

105. The method according to claim 104, wherein:
the one or more ultrasound transducers comprises a plurality of ultrasound transducers,
placing the housing on the portion of skin of the subject comprises placing on the portion of skin of the subject a housing including:
at least first and second support structures configured to be placed in contact with a surface of the skin,
the first support structure having a first concave surface, the first support structure being coupled to one or more first ultrasound transducers of the plurality of ultrasound transducers, and
the second support structure having a second concave surface that faces the first concave surface, the second support structure being coupled to one or more second ultrasound transducers of the plurality of ultrasound transducers, and
the method further comprises:
drawing the portion of skin and underlying tissue between the first and second support structures, wherein
configuring the one or more ultrasound transducers to induce the implosion wave in the target region comprises driving the first and second ultrasound transducers to induce the implosion wave in the target region under the portion of the skin.

106. The method according to claim 105, wherein driving the first and second ultrasound transducers to induce the implosion wave comprises driving the first and second ultrasound transducers to induce a series of positive-pressure implosion waves in the target region.

107. The method according to claim 105, wherein driving the first and second ultrasound transducers to induce the implosion wave comprises driving the first and second ultrasound transducers to induce cavitation as a result of the implosion wave.

108. The method according to claim 105, wherein driving the first and second ultrasound transducers to induce the implosion wave comprises driving the first and second ultrasound transducers to transmit at least one shock wave in the target region.

109-110. (canceled)

111. The method according to claim 105, wherein driving the first and second ultrasound transducers to induce the implosion wave comprises treating soft tissue of the subject.

112-115. (canceled)

116. The method according to claim 105, further comprising:
during a first period, driving the first and second ultrasound transducers to induce at least one series of negative pressure pulses of acoustic energy in the target region; and
during a second period subsequent to the first period, driving the first and second ultrasound transducers to induce at least one series of positive pressure pulses of acoustic energy in the target region; and wherein
driving the first and second ultrasound transducers to induce the implosion wave in the target region comprises driving the first and second ultrasound transducers to induce the implosion wave while substantially inhibiting cavitation in tissue of the subject, by directing the pressure pulses from the first and second ultrasound transducers to the target region during the first and second periods.

117-126. (canceled)

127. The method according to claim 105, further comprising:
transmitting toward the target region treatment energy from the first ultrasound transducer,
transmitting toward the target region treatment energy from the second ultrasound transducer,
receiving through-transmitted energy by at least one ultrasound transducer selected from the group consisting of: the first ultrasound transducer and the second ultrasound transducer, and
monitoring a change in a parameter of tissue in the target region, responsively to the received energy.

128-133. (canceled)

134. The method according to claim 127, further comprising:
receiving scattered waves from the tissue in the target region by at least one ultrasound transducer selected from the group consisting of: the first ultrasound transducer and the second ultrasound transducer,
monitoring acoustic properties of the tissue responsively to the receiving, and
generating a temperature map based on the monitoring of the acoustic properties of the tissue.

135-137. (canceled)

138. The method according to claim 134, wherein monitoring comprises differentiating between types of tissue in the target region.

139. The method according to claim 138, wherein the tissue includes adipose tissue and non-adipose tissue, and wherein the method further comprises:
driving the first and second ultrasound transducers to selectively induce the implosion wave in the adipose tissue, and
damaging the adipose tissue responsively to the inducing.

140-144. (canceled)

145. A method, comprising:
placing on skin of a subject a housing including:
at least first and second support structures configured to be placed in contact with a surface of the skin,
the first support structure having a first concave surface, the first support structure being coupled to two or more first electrodes, and
the second support structure having a second concave surface that faces the first concave surface, the second support structure being coupled to two or more second electrodes;
drawing at least a portion of the skin and underlying tissue between the first and second support structures; and
driving the first and second electrodes to induce an implosion wave in a target region under the portion of the skin.
